(19) United States
(12) Patent Application Publication
(10) Pub. No.: US 2024/0182425 A1
(43) Pub. Date: Jun. 6, 2024

(54) NOVEL COMPOUNDS PHOTO-CROSSLINKED BY VISIBLE LIGHT AND USES THEREOF

(71) Applicant: SEOUL NATIONAL UNIVERSITY R&DB FOUNDATION, Seoul (KR)

(72) Inventor: Hyun Woo RHEE, Seoul (KR)

(21) Appl. No.: 17/784,298

(22) PCT Filed: Apr. 13, 2022
(86) PCT No.: PCT/KR2022/005350

§ 371 Date: Jun. 10, 2022

(30) Foreign Application Priority Data
Apr. 13, 2021 (KR) 10-2021-0047647
Nov. 5, 2021 (KR) 10-2021-0151263

(51) Int. Cl. C07D 221/14; G01N 33/68

(52) U.S. Cl. CPC C07D 221/14; G01N 33/6845; G01N 2333/165

(57) ABSTRACT

The present invention relates to novel compounds that are photo-crosslinked by visible light and spatiotemporal proximity photo-crosslinking by visible light activation (spotlight) using the same. When a target nucleic acid or target protein is bound to the novel compounds by the property of being photo-crosslinked to the proximal protein or peptide by irradiation with visible light and it is irradiated with visible light, the novel compounds according to the present invention can be photo-crosslinked with a protein or peptide that physically interacts with the target protein or target nucleic acid, and thus, there are advantages in that it is possible to overcome diffusive labeling, which is a limitation of the conventional proximity labeling technology, by photo-crosslinking by visible light irradiation, which is a safe method for a living body, and to improve the accuracy of protein interactome identification.

### TECHNICAL FIELD

The present invention relates to novel compounds that are photo-crosslinked by visible light and uses thereof, and more specifically to novel compounds that are photo-crosslinked by visible light and spatiotemporal proximity photo-crosslinking by visible light activation (spotlight) using the same.

### BACKGROUND ART

Most proteins physically interact with other proteins and form macromolecular complexes to perform their biological functions. However, only a few methods are available to reliably reveal unknown protein-protein interaction (PPI) network in living cells. Recently, proximity labeling (PL) technology was developed based on in situ-generated reactive species using various genetically encodable enzymes such as ascorbate peroxidase (APEX) or various biotin ligases such as BioID or TurboID, and the reactive species generated by APEX or BioID/TurboID are covalently conjugated to proximal proteins near the enzymes in living cells and analyzed using mass spectrometry after cell lysis. This method has become increasingly popular in cell biology and has revealed local proteome information in diverse subcellular compartments within a live cellular context.

The labeling radius of APEN BioID/TurboID and other proximity labeling tools (e. T-Rex, PhotoPPI) is estimated to range from approximately 10 nm to a few hundred nanometers, depending on the lifetime of the reactive species and local protein concentration in live cells. This labeling radius is suitable for sub-compartmental proteomic mapping; however, this labeling radius is rather diffusive for the identification or interaction analysis of the physical interaction partners (i.e., interactome analysis) of proteins of interest (POO.

Meanwhile, in the case of interactome mapping, photo-crosslinking reactions have been utilized to capture the physical interactome, and this method utilizes aryl azide, diazo or diazitine moieties that can be converted into reactive nitrene or carbene species by ultraviolet (UV) light activation. Since these species usually have a short lifetime (T_{1/2}<2 ns) in an aqueous solution, they can conduct N—H or C—H insertion reactions with physically interacting proteins. Photo-crosslinking methods using unnatural amino acids (UAAs) such as photo-methionine or photo-leucine or those containing diazirine moieties enable photo-crosslinking reaction of the proteins into which UAAs are incorporated via the translational machinery in live cells.

However, this method faces two critical issues: (i) toxicity from proteome-wide incorporation of UAAs and (ii) UV light irradiation that has detrimental consequences for a live system. To overcome the issues of current proximity labeling and photo-crosslinking techniques, photo-crosslinking by visible light can be attempted, but since most currently known visible light active moieties that can be used (e.g., benzoyl azide, vinyl azide) are unstable in physiological systems (Brachet, E, et al., Chem Sci. 2015, 6, 987-992; Borra, S. et at, Org Biol. Chem, 2019, 17, 5971-5981), there is a limitation in grafting the same to intracellular proximity labeling.

Naphthyl azide-based AzNP (4-azido-N-ethy-1,8-naphthalimide) is (1) very stable in an aqueous solution and has been widely used in a hydrogen sulfide sensor, (2) has been used for fluorescence imaging of biomolecules modified to alkyne in living mammalian cells by emitting strong green fluorescence, and (3) upon UV irradiation, AzNPs show good photo-crosslinking ability to be converted into aryl nitrene species capable of covalently bonding to proteins. However, the visible light-induced photo-crosslinking activity of AzNP in living cells has not been confirmed so far.

The inventors of the present invention have made extensive efforts to develop a method for improving the conventional technique of diffusive labeling and accurately identifying an interactome physically interacting with a target protein in a living cell as a method for identifying an interactome physically interacting with the target protein in a cell, and as a result, the inventors of the present invention newly synthesized compounds that can be photo-crosslinked to the proximal protein by visible light, and the present invention was completed by confirming that when the compounds are used, the accuracy of the protein-protein interaction analysis method can be increased by photo-crosslinking to the proximal protein without toxicity to living cells by irradiation with visible light, thereby improving the diffusion labeling characteristic, which is a limitation of the conventional proximity labeling technology.

### DISCLOSURE

### Technical Problem

An object of the present invention is to provide novel compounds that can be crosslinked by visible light and uses thereof.

### Technical Solution

In order to achieve the above object, the present invention provides a compound represented by [Chemical Formula 1] below:

[Chemical Formula 1]

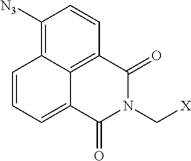

wherein in [Chemical Formula 1] above,
X is C, N, O, S, Si or represented by [Chemical Formula 2] below,

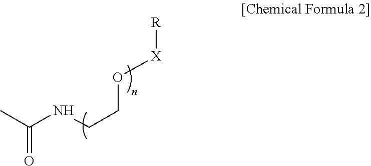

wherein in [Chemical Formula 2] above,
n is an integer of 0 to 10,
X is straight-chain or branched —(CH₂)m-, H or halogen, and m is an integer of 1 to 20, and
R is H, halogen or —CH₃.

In the present invention, the compound may be a compound represented by [Chemical Formula 3] below:

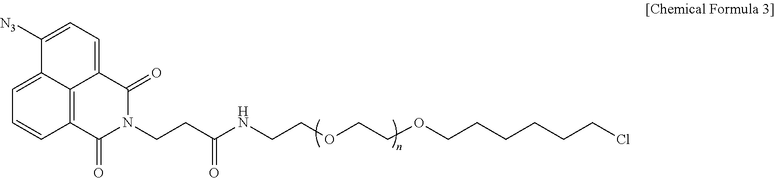

wherein in [Chemical Formula 3] above, n is an integer of 1 to 5.

In the present invention, the compound may have protein-binding ability.

In the present invention, the compound bound to a target protein or target nucleic acid may be photo-crosslinked to a protein or peptide that interacts with the target protein or target nucleic acid.

In the present invention, the target protein may be an antibody or peptide.

In the present invention, the target nucleic acid may be DNA or RNA.

In the present invention, the compound may be photo-crosslinked to a protein or peptide that interacts with the target protein or target nucleic acid by irradiation with visible light.

In the present invention, the radius of the photo-crosslinking may be 0.01 to 1 nm.

In addition, the present invention provides a method for photo-crosslinking a target protein or target nucleic acid with an interacting protein or peptide in proximity, including the steps of:
(a) treating the compound bound to a target protein or target nucleic acid in a sample including a protein or peptide interacting with the target protein or target nucleic acid; and
(b) processing visible light.

In the present invention, the target protein or target nucleic acid may be conjugated to the compound through (i) an internally existing amine group or an externally introduced amine group, or (ii) an internally existing thiol group or an externally introduced thiol group.

In the present invention, the target protein or target nucleic acid may be conjugated with a protein tag

In addition, the present invention provides a method for identifying an interacting protein of a target protein, including the steps of:
(a) expressing a target protein in a cell;
(b) treating the cell with the compound;
(c) irradiating visible light to the cell; and
(d) lysing the cell to identify the protein that is photo-crosslinked with the compound as an interacting protein of the target protein.

In the present invention, the target protein may be conjugated with an epitope peptide tag.

In the present invention, in step (d), the target protein may be precipitated with an epitope peptide-specific antibody to identify the protein that is photo-crosslinked with the compound as an interacting protein of the target protein.

In the present invention, step (d) may be identified by mass spectrometry.

### Advantageous Effects

The novel compounds according to the present invention can be photo-crosslinked with a protein or peptide that physically interacts with a target protein or target nucleic acid by the property of being photo-crosslinked to the proximal protein or peptide by irradiation with visible light by binding to the target protein or target nucleic acid, and thus, there are advantages in that it is possible to overcome diffusive labeling, which is a limitation of the conventional proximity labeling technique, by photo-crosslinking by visible light irradiation, which is a safe method for a living body, and to improve the accuracy of protein interactome identification.

### DESCRIPTION OF DRAWINGS

FIG. 1 shows the synthesis processes of two AzNP-conjugated HaloTag ligands (VL1 and VL2) and four control ligands (UL1-UL4) according to the present invention.

FIG. 2A is mimetic diagrams showing proximity labeling, the proximity labeling according to the present invention and a conventional photo-crosslinking reaction.

FIG. 2B is a table comparing the characteristics of proximity labeling, the proximity labeling according to the present invention and a conventional photo-crosslinking reaction.

FIG. 2C shows the photo-activation reaction mechanism of the AzNP moiety.

FIG. 2D shows the probe synthesis reaction of the HaloTag-conjugated version of the AzNP moiety.

FIG. 2E shows the X-ray structures of co-crystallized HaloTag and VL1.

FIG. 2F is a mimetic diagram showing VL1-mediated FKBP-FRB photo-crosslinking according to rapamycin treatment under blue LED light illumination.

FIG. 2G shows the Western blot results showing whether KBP-FRB mediated by VL1, VL2 and UL2 is photo-crosslinked according to rapamycin treatment under blue LED light illumination. The photo-crosslinked products of FKBP25-V5-HaloTag and EGFP-FRB are indicated by red asterisks, and non-photo-crosslinked FKBP25-V5-HaloTag and EGFP-FRB are indicated by blue asterisks.

FIG. 3A is mimetic diagrams showing proximity photo-crosslinking using a phenylazide-conjugated HaloTag ligand.

FIG. 3B shows the reaction mechanism of UV-activated photo-crosslinkers (i.e., phenylazide, diazirine).

FIG. 3C shows the chemical structures (UL1, UL2, UL3, UL4) of UV-activated para-azidophenyl and diazirine conjugated probes.

FIG. 3D shows the co-crystal structures of HaloTag and UL2.

FIG. 3E shows mimetic diagrams of UL probe-mediated FKBP-FRB photo-crosslinking in the presence of rapamycin and UV light.

FIG. 3F shows the photo-crosslinking test results of EGFP-FRB and FKBP25-V5-HaloTag using various UV-activated photo-crosslinking probes. EGFP-FRB and FKBP25-V5-HaloTag crosslinked products are indicated by red asterisks, and uncrosslinked FKBP25-V5-HaloTag and EGFP-FRB are indicated by blue asterisks.

FIG. 3G shows the Western blot results of UL2-mediated photo-crosslinking of FKBP25-V5-HaloTag and EGFP-FRB using rapamycin (100 nM) and UV light illumination (10 min) in living cells. EGFP-FRB and FKBP25-V5-HaloTag crosslinked products are indicated by red asterisks, and uncrosslinked FKBP25-V5-HaloTag and EGFP-FRB are indicated by blue asterisks.

FIG. 3H shows the Western blot results of crosslinked FKBP25-V5-HaloTag and EGFP-FRB according to immunoprecipitation of FKBP25-V5-HaloTag via C-terminal-tagged biotinylated receptor peptide (AP). In the cell lysate, AP was biotinylated by the addition of biotin ligase (BirA), biotin and ATP, and the biotinylated protein was concentrated by streptavidin magnetic beads. In the eluted fraction, crosslinked EGFP-FRB and FKBP25-V5-HaloTag products (red asterisks) and uncrosslinked EGFP-FRB (blue asterisks) were all observed in the rapamycin-treated samples. Anti-EGFP antibody was used for the detection of EGFP-FRB.

FIG. 3I is the Coomassie staining results of purified FKBP12-HaloTag crosslinking using purified FRB-EGFP in lane 6 showing that FKBP12-HaloTag was crosslinked with FRB-EGFP by UL2 probe after rapamycin pretreatment.

FIG. 4A is mimetic diagrams showing the sequential reactions of VL1-mediated photo-crosslinking and biotin labeling of FKBP12-V5-HaloTag-TurboID in living cells.

FIG. 4B is the results of observation of the expressions of FKBP12-V5-HaloTag-TurboID and EGFP-FRB in HEK293T cells treated with rapamycin (100 nM) or a vehicle for 1 hour in confocal images. Cells were fixed and permeabilized after incubation with biotin (50 μM) for 30 minutes. FKBP25-V5-HaloTag-TruboID was visualized with anti-V5 antibody and anti-mouse Alexa Fluor 568. Biotinylated proteins were visualized with SA-647 antibody after fixation and permeabilization. The green channel shows the EGFP signal. Scale bar=10 μm.

FIG. 4C shows the Western blot results of VL-1 mediated FKBP12-V5-HaloTag and EGFP-FRB photo-crosslinking under blue LED light. Photo-crosslinked products are indicated by red asterisks and uncrosslinked monomers are indicated by blue asterisks.

FIG. 4D shows the anti-Flag concentration results of FKBP12-HaloTag-V5-TruboID-Flag in the samples from lanes 3 and 4 of FIG. 4C. VL-1 mediated crosslinked products (red asterisks) in anti-V5 and anti-GFP blots are shown as biotinylated proteins in the SA-HRP Western blots. Uncrosslinked monomers are indicated by blue asterisks.

FIG. 5A shows the fluorescence images of HaloTag-conjugated POIs and VLs localized in various cells. Scale bar=10 μm. VL1 fluorescence was observed in the GFP channel of a confocal microscope (excitation wavelength=488 nm).

FIG. 5B is the Western blot analysis results using an anti-V5 antibody against VL-1 mediated photo-crosslinking products of various Halotag-V5-POIs. Photo-crosslinked products are indicated by red asterisks and uncrosslinked POI-HaloTag proteins are indicated by blue asterisks.

FIG. 6A is mimetic diagrams showing the process of monitoring the stress granule formation of G3BP1-HaloTag with VL1 fluorescence under various stress conditions (e.g., heat, chemical treatment or viral infection).

FIG. 6B shows the results of confocal images of G3BP1-EBFP-V5-Halotag treated with 50011M arsenite or VL1-treated according to heat shock (43° C.) for 1 hour. The EGFP fluorescence of G3BP1-EBFP-V5-Halotag was observed with BFP channel. VL1 fluorescence was observed with GFP channel, and anti-V5 immunofluorescence was visualized with a secondary antibody conjugated to Alexa Fluor 647. Scale bar=10 μm.

FIG. 6C shows the Western blot results of the VL1-mediated crosslinked product of G3BP1-EGFP-HaloTag under various stress conditions of FIG. 6b. An ophthalmic sample not incubated with VL1 was used as a negative control.

FIG. 6D is the results of line scan analysis of the band intensity of the crosslinked product of FIG. 6B.

FIG. 7A is the results of confocal imaging of N-HaloTag (^{SARs-CoV-2}N-v5-HaloTag).

FIG. 7B is the Western blot results of the VL-mediated crosslinked product of N-HaloTag (indicated by red asterisks). Uncrosslinked N-HaloTags are indicated by blue asterisks.

FIG. 7C schematically shows the VL1-crosslinking proteins of N-HaloTag and HaloTag. The table shows the mass spectrometry protein results tested in triplicate. The intensity of color indicates the mass intensity of peptides per VL-crosslinked protein identified with N-HaloTag or HaloTag.

FIG. 7D shows the normalized mass intensities of the 14 proteins of Group I of FIG. 7C. RBP is shown in yellow and non-RBP is shown in blue.

FIG. 7E shows the Volcano plots of Groups II to IV of FIG. 7C showing statistically significant concentration of VL1-crosslinked proteins of N-Halotag (8 proteins, Group II) with respect to VL1-crosslinked proteins of HaloTag alone.

FIG. 7F shows the confocal microscopy images of HAloTag-G3BP1 and VL1 in 293T cells. Scale bar=10 μm.

FIG. 7G shows the confocal microscopy images of N-GFP co-expressed with HaloTag-V5-G3BP1 in 293T cells. HaloTag-V5-G3BP1 was observed with an anti-V5 antibody (AF568-conjugated secondary antibody, red fluorescence channel), VL1 and N-GFP with GFP channel. Arrows indicate stress granule formation. Scale bar=10 μm.

FIG. 7H is the anti-V5 and anti-GFP Western blot results of the VL1-crosslinked products of HaloTag-V5-G3BP1 and N-GFP. The photo-crosslinked product is indicated by a red asterisk, and the uncrosslinked N-HaloTag protein is indicated by a blue asterisk.

FIG. 7I is the anti-V5 and anti-GFP Western blot results of the VL1-crosslinked products of N-HaloTag and G3BP1-GFP. The photo-crosslinked product is indicated by a red asterisk, and the uncrosslinked N-HaloTag protein is indicated by a blue asterisk.

FIG. 8A shows the complete binding assay of VL1 using biotin-HTL in N-HaloTag-expressing HEK293T cells. After incubation with VL1 (10 μM) for 60 minutes, biotin-HTL (10 μM) was treated for 60 minutes. Biotin-HTL can label the empty N-HaloTag protein with or without VL1 pretreatment, and streptavidin-HRP can detect this population in Western blot analysis.

FIG. 8B is a bar graph showing the band intensities of biotin-HTL labeled signals.

FIG. 8C is the comparison results of the photo-crosslinking efficiency of VL and UL probes for ^{SARS-CoV-2}N-HaloTag under blue LED illumination. Anti-V5 antibody was used for the detection of N-HaloTag.

FIG. 8D is a bar graph showing the band intensities of the photo-crosslinked products of FIG. 8C.

FIG. 8E is the comparison results of photo-crosslinking efficiency of VL and UL probes for ^{SARS-CoV-2}N-HaloTag under UV illumination. Anti-V5 antibody was used for the detection of N-HaloTag.

FIG. 8F is a bar graph showing the band intensities of the photo-crosslinked products of FIG. 8E.

FIG. 8G shows the VL1-mediated photo-crosslinked products of N-HaloTag over time during blue LED illumination (0 to 10 minutes).

FIG. 8H is a bar graph showing the band intensities of the photo-crosslinked products of FIG. 8G.

FIG. 9A shows the flow chart of a sample preparation process for mass spectrometry of the crosslinked samples according to the present invention.

FIG. 9B is mimetic diagrams showing the processing conditions of each sample used for mass analysis of the crosslinked samples according to the present invention.

FIG. 10A is the results of confirming protein-protein interaction by VL-crosslinking analysis.

FIG. 10B is the results of comparing the crosslinking effect according to the linker length of HaloTag-G3BP1.

### MODES OF THE INVENTION

Hereinafter, exemplary embodiments will be provided to describe the present specification in detail. However, the exemplary embodiments according to the present specification may be modified in various other forms, and the scope of the present specification is not to be construed as being limited to the exemplary embodiments described below. The exemplary embodiments of the present specification are provided to more completely explain the present specification to those of ordinary skill in the art.

In the present invention, novel compounds that can specifically bind to an amine group or a thiol group and be photo-crosslinked with a proximal protein by blue light were synthesized.

Accordingly, the present invention relates to a compound represented by [Chemical Formula 1] below in one aspect:

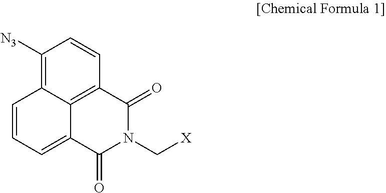

wherein in [Chemical Formula 1] above,
X is C, N, O, S, Si or represented by [Chemical Formula 2] below,

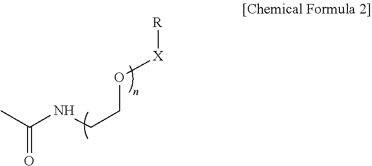

wherein in [Chemical Formula 2] above,
n is an integer of 0 to 10,
X is straight-chain or branched —(CH₂)m-, H or halogen, and m is an integer of 1 to 20, and
R is H, halogen or —CH₃.

In the present invention, the compound may be represented by Chemical Formula 1′ below.

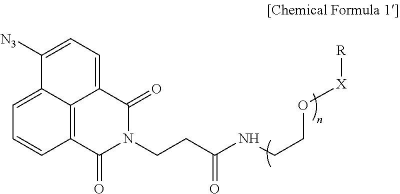

wherein in [Chemical Formula 1′] above,
n is an integer of 0 to 10,
X is straight-chain or branched —(CH₂)m-, H or halogen, and m is an integer of 1 to 20, and
R is H, halogen or —CH₃.

In [Chemical Formula 1′] according to the present invention, preferably, n may be a natural number of 2 to 5, and more preferably, n may be 2 or 3.

In the present invention, preferably, X may be straight-chain or branched —(CH₂)m, and m may be an integer of 1 to 10. More preferably, X may be straight-chain —(CH₂)m-, and m may be an integer of 5 to 10.

In the present invention, preferably, R may be halogen, and more preferably, chlorine (Cl).

In the present invention, the compound may be characterized as a compound represented by [Chemical Formula 3] below, but is not limited thereto:

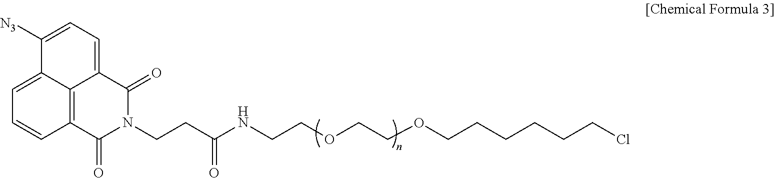

wherein in [Chemical Formula 3] above, n is an integer of 1 to 5.

In [Chemical Formula 3] according to the present invention, preferably, n may be 1 or 2. When n is 1, it may mean ligand VL1, and when n is 2, it may mean ligand VL2.

In the present invention, the compound may be characterized in that it is bound to a target substance to identify a proximal molecule (e.g., an interacting drug, an interacting protein and an interacting peptide present in close proximity) that interacts with the target substance (e.g., a target nucleic acid or a target protein).

In the present invention, the target nucleic acid may be DNA or RNA, but is not limited thereto, and the target protein may be an antibody or peptide, but is not limited thereto.

In one aspect, the compound may be characterized in that it is directly bound to a target substance, but is not limited thereto.

The compound may be an AzNP having an NHS ester group capable of reacting with an amine group of the target substance, by introducing an amine group present in the target protein or an amine group from the outside when synthesizing the target nucleic acid.

In addition, the compound may be an AzNP having a maleimide group capable of reacting with the target substance, by introducing a cysteine group present in the target protein or a thiol group from the outside when synthesizing the target nucleic acid.

In another aspect, the compound may be characterized in that it is bound through a protein tag, but is not limited thereto. That is, in some aspects, the compound may be characterized in that it specifically binds to a protein tag, and the protein tag may be Halotag, SNAP-tag or CLIP-tag, but is not limited thereto.

In the present invention, when the ligand is VL1 or VL2, the protein tag may be preferably Halotag. The ligand VL1 has a shorter length than VL2 and may bind to the protein tag faster than VL2.

In the present invention, the compound may be characterized in that it is photo-crosslinked to a proximal molecule that interacts with the target substance.

That is, the compound bound to the target protein or target nucleic acid may be characterized by photo-crosslinking to a protein or peptide that interacts with the target protein or target nucleic acid.

In the present invention, the target protein may be characterized as an antibody or peptide, but is not limited thereto.

In the present invention, the target nucleic acid may be characterized as DNA or RNA, but is not limited thereto.

In the present invention, the compound may be characterized by photo-crosslinking to a protein or peptide that interacts with the target protein or target nucleic acid by irradiation with visible light.

In one aspect, when the target substance is treated with living cells or protein mixture and treated with visible light, photo-crosslinking may be induced with a protein or peptide that interacts with the target substance in the cells or protein mixture.

In one aspect, the compound may be characterized in that it photo-crosslinks to a protein that interacts with a target protein fused to the protein tag.

In the present invention, the visible light may be used without limitation as long as it is in the range of electromagnetic waves visible to the human eye, but preferably, it may be light at 400 to 700 nm, and more preferably, blue light at 450 to 500 nm.

In the present invention, the radius of the photo-crosslinking may be characterized to be 0.01 to 1 nm, but is not limited thereto.

In an aspect of the present invention, the length of the PEG linker of [Chemical Formula 3] above may determine the direction of active species on the surface of a protein tag (e.g., HaloTag), thereby affecting crosslinking efficiency for a specific interaction partner. Therefore, this feature of the present invention, which has a position-dependent crosslinking efficiency due to the inherently short labeling radius, may be a limiting factor in covering all of the complete physical interactions of the target protein, but from another point of view, due to this structural feature of the present invention, the present invention has the advantage of being able to identify only the protein interacting with the target protein by crosslinking.

In another aspect, the present invention relates to a method for photo-crosslinking a target protein or target nucleic acid with an interacting protein or peptide in proximity, including the steps of:
(a) treating the compound bound to a target protein or target nucleic acid in a sample including a protein or peptide interacting with the target protein or target nucleic acid; and
(b) processing visible light.

In the present invention, the target protein or target nucleic acid may be characterized in that it is conjugated to the compound through (i) an internally existing amine group or an externally introduced amine group, or (ii) an internally existing thiol group or an externally introduced thiol group.

In the present invention, in another aspect, the target protein or target nucleic acid may be characterized in that it is conjugated with a protein tag, but is not limited thereto.

In another aspect, the present invention relates to a method for identifying an interacting protein of a target protein, including the steps of:
(a) expressing a target protein in a cell;
(b) treating the cell with the compound;
(c) irradiating visible light to the cell; and
(d) lysing the cell to identify the protein that is photo-crosslinked with the compound as an interacting protein of the target protein.

In the present invention, the target protein may be characterized in that it is conjugated with an epitope peptide tag. By tagging the target protein with an epitope peptide, the target protein may be immunoprecipitated.

In one aspect, the target protein may be selected from the group consisting of a Flag tag, a Myc tag, an HA tag, an Avi tag and a V5 tag, but is not limited thereto.

In the present invention, the target protein may be isolated by immunoprecipitation with magnetic or agarose beads loaded with an antibody capable of binding to an epitope peptide tag, but is not limited thereto.

In another aspect, the target protein is characterized in that it binds streptavidin, avidin or biotin and is isolated by sedimentation with magnetic or agarose beads loaded with an analog of streptavidin or an analog of avidin reversibly binding to biotin, but is not limited thereto.

Meanwhile, in the present invention, the direct interacting partner of a target protein may be selectively and accurately identified in a living cell as compared to a conventional proximity labeling method, and through this, it is possible to confirm a molecular network of the target protein in a living cell.

TurboID has been utilized for interactome mapping in various experiments, but due to the rather diffuse labeling properties of biotin-AMP, recent proximity labeling methods (e.g., BioID, TurboID and APEX) have additionally required “a filtration approach” that compares data to the biotinylated proteins of cytoplasmic TurboID (e.g. TurboID-NES). However, these filtered data still cannot selectively reflect only direct binding partners, but rather only provide an ambiguous “contour” of the proximal protein compared to the cytoplasmic background, and there was a limitation in that it was not possible to decipher whether the protein selected from this data was a true interacting partner of the target protein.

In order to overcome this problem, considering that the photo-crosslinking (Spotlight) reaction according to the present invention may be orthogonal to the amide coupling-based biotinylation reaction of TurboID, in the present invention, TurboID was applied to the same target protein in addition to the photo-crosslinking reaction according to the present invention, and as a result, it was confirmed that the physical interacting protein of the target protein may be biotinylated together with photo-crosslinking by the compound of the present invention.

Accordingly, in another aspect, the present invention relates to a method for identifying an interacting protein of a target protein, including the steps of:
(a) expressing a target protein in a cell;
(b) treating the cell with the compound;
(c) irradiating visible light to the cell; and
(d) lysing the cell to identify the protein that is photo-crosslinked with the compound as an interacting protein of the target protein.

In the present invention, the target protein in step (a) may be further fused with biotin ligase.

In one aspect, the protein tag may be fused to a target protein by a linker.

In the present invention, the linker may be an amide linker, but is not limited thereto.

Meanwhile, as a result of an experiment using HaloTag-G3BP1 to determine if the generation efficiency of the crosslinked product is affected by the linker in the present invention, it was confirmed that the HaloTag position at the N-terminus of G3BP1 may have some flexibility to access the surface of the ^{SARS-CoV-2}N protein, and thus is not substantially affected by the length of the peptide linker between the HaloTag and G3BP1.

Accordingly, a linker known in the art, which is commonly used for linkage between proteins/proteins, proteins/peptides or peptides/peptides, may be used without limitation to bind HaloTag and a target protein in the present invention.

In the present invention, the compound may be characterized in that it specifically binds to a protein tag, and the protein tag may be Halotag, SNAP-tag or CLIP-tag, but is not limited thereto. In the present invention, the protein tag may be preferably Halotag.

In the present invention, the biotin ligase may be characterized as BirA, BioID or TurboID, but is not limited thereto. In the present invention, the biotin ligase may preferably be TurboID.

In the present invention, the step (d) may be characterized in that it further includes a step of isolating the target protein and the photo-crosslinked protein from the lysed cells, and the step of isolating the photo-crosslinked protein may be characterized by fusing an epitope peptide tag (e.g., a Flag tag, a Myc tag, an HA tag, an Avi tag, a V5 tag, etc.) to the target protein so as to immuneprecipitate the target protein, and isolating the target protein by using magnetic or agarose beads loaded with an antibody specifically binding to the epitope peptide tag, but the present invention is not limited thereto.

In another aspect, the step of isolating the photo-crosslinked protein may be characterized in that streptavidin, avidin or biotin is added to the target protein, and the photo-crosslinked protein is isolated using magnetic or agarose beads loaded with an analog of streptavidin reversibly binding to the streptavidin, avidin or biotin or an analog of avidin reversibly binding to biotin, but the present invention is not limited thereto.

In the present invention, step (d) may be characterized in that it further includes a step of digesting the isolated protein with a hydrolase, but is not limited thereto.

In the present invention, the hydrolase may be characterized in that it is selected from the group consisting of trypsin, arginine C (Arg-C), aspartic acid N (Asp-N), glutamic acid C (Glu-C), lysine C (Lys-C), chymotrypsin, proteinase K and pronase, but is not limited thereto.

In the present invention, step (d) may be characterized in that the interacting protein is identified by mass spectrometry, but is not limited thereto.

In the present invention, the mass spectrometer for mass spectrometry may be characterized in that it is selected from the group consisting of LTQ-FT, Orbitrap, Triple-Tof, Q-Tof, Tof-Tof and Q Exactive, but is not limited thereto.

In the present invention, in step (d), the protein that is biotinylated by a control fusion protein to which biotin ligase and a protein tag are fused in the cell may characterized in that it is excluded from the interacting protein of the target protein, but is not limited thereto.

In the present invention, in an exemplary embodiment related to the above aspect, in order to confirm whether each step of the experiment (e.g., photo-crosslinking and streptavidin concentration) operates as expected, mass analysis was performed on 8 samples of ^{SARS-CoV-2}N-HaloTag-AP and HaloTag-AP under four control conditions (+/−VL1, +/−BirA), but in order to identify the interacting protein of the target protein, it may be sufficient to compare the streptavidin-concentrated sample of the VL1-crosslinked POI-HaloTag (POI-HaloTag-AP/+VL1/+BirA) with the VL1-crosslinked free HaloTag (HaloTag-AP/+VL1/+BirA), and in this case, the free HaloTag-crosslinked protein may provide background protein information for other experiments for identifying various types of interacting proteins of the target protein by applying the present invention.

Further, in the present invention, it was confirmed that the location of the target protein in living cells may be identified based on the characteristic that the compound can penetrate various intracellular organelles and exhibit green fluorescence, and it is possible to verify whether the target protein interacts with an interacting protein candidate group based on imaging.

Accordingly, in another aspect, the present invention relates to a method for identifying an interacting protein of a target protein, including the steps of:
(a) expressing a target protein to which a protein tag is fused in a cell;
(b) treating the cell with the compound;
(c) irradiating visible light to the cell; and
(d) identifying the intracellular location of the target protein by green fluorescence.

According to the present invention, after step (a) in the above method, the intracellular movement of the target protein may be easily observed under a stress situation by inducing stress (change in acidity, change in temperature, etc.).

In another aspect, the present invention relates to a method for verifying interaction with a target protein and an interactome thereof, including the steps of:
(a) expressing a target protein to which a protein tag is fused and an interactome thereof in a cell;
(b) treating the cell with the compound;
(c) irradiating visible light to the cell; and
(d) confirming whether the target protein and the interactome are co-localized in the cell.

In the present invention, the intracellular location of the target protein may be identified by green fluorescence, and the interactome may be identified by a fluorescence different from the green fluorescence, and by observing the co-localization of the two types of fluorescence, it will be possible to determine whether there is an interaction between the protein and the interactome thereof.

In the present invention, fluorescence for identifying the interactome may be identified by labeling the interactome-specific antibody with fluorescence or engineering the interactome to be expressed together with fluorescence in a cell.

According to the present invention, after step (a) in the above method, it is possible to easily determine how the target protein and the interactome thereof interact in the before and after stress situations by inducing stress (change in acidity, change in temperature, etc.).

In some aspects of the present invention, the protein may be proteins or virus proteins distributed in various organelles, including Lamin-AC (inner nuclear membrane), p80-coilin (Cajal body), HNRNPD (SAM68 body), MRPL12 (mitochondrial matrix), Tom20 (outer mitochondrial membrane), SEC61B (ER membrane) and G3BP1 (stress granule). For example, the protein may be a nucleocapsid (N) protein.

According to an exemplary embodiment of the present invention, the virus may be a mammalian infection virus, and preferably, SARS-CoV-2 virus. The mammal may be a mouse, hamster, dog, cat, pig, goat or primate, including humans.

It can be seen that the compound of the present invention is activated by irradiation with visible light to effectively have photo-crosslinking ability, and in particular, it is remarkable that in the present invention, the effect was confirmed by using a blue LED home lamp (about 36 W) in all photo-crosslinking experiments using living cells as well as in an in-test tube experiment using VL1. That is, the compound of the present invention may be effectively photo-crosslinked in low-intensity visible light illumination, suggesting that the present invention may be utilized in the in vivo experiments using living animals as well as the in vitro experiments using living cells.

Meanwhile, in a living cell experiment, photo-crosslinked products of the HaloTag protein were produced within 1 minute under blue LED illumination, which was confirmed at a much faster rate than non-targeted in vitro photo-crosslinking with bovine serum albumin, which is interpreted to be due to a macromolecule-dense environment of living cells.

The above-described results support that the spatiotemporal proximity photo-crosslinking by visible light activation (spotlight) may accurately capture transient interacting partners effectively under physiological conditions.

Hereinafter, the present invention will be described in more detail through examples. These examples are only for illustrating the present invention, and it is apparent to those of ordinary skill in the art that the scope of the present invention is not to be interpreted as being limited by these examples.

### Example 1. Synthesis of HaloTag-Specific AzNP Ligands

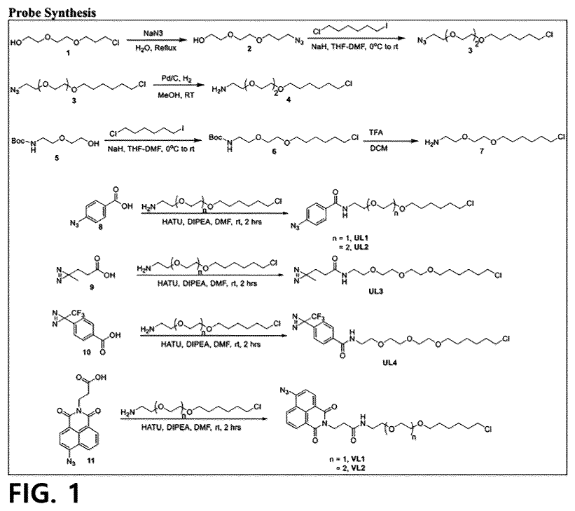

AzNP-conjugated HaloTag ligands (VL1 and VL2) were synthesized with two linker sizes. VL1 showed a fairly strong absorbance in the visible region; Mₘₐₓ=375 nm, extinction coefficient value=2,230 M⁻¹ cm⁻¹. For control experiments, other photo-crosslinkable HaloTag ligands including ultraviolet light-absorbing para-azidophenyl and diazirine moieties were also prepared (UL1-UL4). The reaction schematic is as shown in FIG. 1.

### Synthesis of (1-(2-(2-(2-azidoethoxy)ethoxy)ethoxy)-6-chlorohexane)

NaH (6 mg, 0.15 mmol) was added to a solution of 2-(2-(2-azidoethoxy)ethoxy)ethanol (17.5 mg, compound 2, 0.1 mmol) in DMF at 0° C. After stirring at 0° C. for 30 minutes, 1-chloro-6-iodohexane (18.6 μL, 0.12 mmol) was added. The mixture was stirred for 1 hour, warmed to room temperature and stirred for 2 hours. Ammonium chloride was added to the reaction mixture at 0° C., and after it was extracted with diethyl ether (10 mL×3), it was washed with saturated brine (10 mL), dried over anhydrous Na₂SO₄, filtered and concentrated in vacuum. The crude material was purified by flash chromatography on silica gel to obtain (1-(2-(2-(2-azidoethoxy)ethoxy)ethoxy)-6-chlorohexane) (12 mg, compound 3, yield: 40%).

¹H NMR (400 MHz, Chloroform d) δ(m, 6H), 3.60-3.57 (m, 2H), 3.53 (t, J=6.7 Hz, 2H), 3.46 (t, J=6.6 Hz, 2H), 3.39 (t, J=5.1 Hz, 2H), 1.82-1.73 (m, 2H), 1.60 (p, J=6.8 Hz, 2H), 1.52-1.31 (m, 6H). ¹³C NMR (100 MHz, Chloroform-d) 670.74, 70.72, 70.66, 70.11, 70.04, 50.70, 45.07, 32.56, 29.46, 26.71, 25.43. APCI-MS: m/z calcd. for 293.15, measured for 265.88 [M+H-N2]⁺.

### Synthesis of (2-(2-(2-((6-chlorohexyl)oxy)ethoxy)ethoxy)ethan-1-amine)

After performing 2 cycles of vacuum/H2 to replace the air inside the reaction tube with hydrogen, the mixture of compound 3 (35 mg, 0.12 mmol), 10% Pd/C (3.5 mg 10 wt. % of compound 3) and 2 to 3 drops of 1N HCl in MeOH (0.6 mL) was stirred under a balloon of hydrogen for 2 hours at room temperature. The reaction mixture was filtered using a Celite filter to obtain (2-(2-(2-((6-chlorohexyl)oxy)ethoxy)ethoxy)ethan-1-amine (compound 4) as a quantitative yield.

¹H NMR (400 MHz, DMSO-d6) δ-8.02 (m, 3H), 3.59 (dq, J=6.7, 3.6, 3.1 Hz, 4H), 3.55-3.41 (m, 8H), 3.35 (d, J=2.9 Hz, 2H), 2.90 (t, J=5.3 Hz, 2H), 1.73-1.63 (m, 2H), 1.46 (dd, J=9.9, 4.7 Hz, 2H), 1.40-1.24 (m, 4H). ¹³C NMR (100 MHz, DMSO-d6) δ70.31-69.74 (m), 67.01, 45.82, 38.82, 32.45, 29.47, 26.54, 25.35. APCI-MS: m/z calcd for 267.16, measured for 267.95 [M+H]⁺.

UL1 Synthesis

In order to synthesize an aryl-azide-PEG2 HaloTag ligand (UL1), 4-azidobezoic acid (compound 8, 83 mg, 0.50 mmol) was placed in a 10 mL round bottom flask with HATU (214 mg, 0.56 mmol) and DIPEA (131.3 mg, 1.02 mmol) and dissolved in 2 mL of DCM. The reaction mixture was stirred at room temperature for 30 minutes or until the color turned brown, and subsequently, 2-(2-((6-chlorohexyl)oxy)ethoxy)ethanamine (138 mg, 0.61 mmol) dissolved in 1 mL of DCM was added dropwise to the reaction mixture, and then the reaction was stirred at room temperature for additional 3 hours. After completion of the reaction, the solvent was removed under reduced pressure to obtain a colorless oil, which was purified by silica gel column chromatography (eluent: 40% EtOAc in hexane) to obtain pure UL1 (168 mg, 90%) as a colorless oil.

¹H NMR (400 MHz, CDCl₃) δ(d, J=8.7 Hz, 2H), 7.06 (d, J=8.7 Hz, 2H), 6.67 (br s, 1H), 3.70-3.63 (m, 6H), 3.62-3.57 (m, 2H), 3.52 (t, J=6.7 Hz, 2H), 3.46 (t, J=6.7 Hz, 2H), 1.78-1.70 (m, 2H), 1.61-1.54 (m, 2H), 1.46-1.32 (m, 4H). ¹³C NMR (100 MHz, CDCl₃) δ143.35, 131.19, 128.93, 119.04, 77.16, 71.39, 70.35, 70.12, 69.81, 45.12, 39.84, 38.73, 32.59, 29.55, 26.78, 25.50, HR-ESI-MS: m/z calcd. for 368.1615, measured for 368.1614.

UL2 Synthesis

In order to synthesize an aryl-azide-PEG3 HaloTag ligand (UL2), 4-azidobezoic acid (compound 8, 45 mg, 0.28 mmol) was placed in a 10 mL round bottom flask with HATU (105 mg, 0.28 mmol) and DIPEA (64 mg, 0.58 mmol) and dissolved in 2 ml of DCM. The reaction mixture was stirred at room temperature for 30 minutes or until the color turned brown, and subsequently, 2-(2-(2-((6-chlorohexyl)oxy)ethoxy)ethoxy)ethanamine (68 mg, 0.25 mmol) dissolved in 1 mL of DCM was added dropwise to the reaction mixture, and then the reaction was stirred at room temperature for additional 3 hours. After completion of the reaction, the solvent was removed under reduced pressure to obtain a colorless oil, which was purified by silica gel column chromatography (eluent: 60% EtOAc in hexane) to obtain UL2 (98 mg, 95%) as a colorless oil.

¹H NMR (400 MHz, Acetone) 6 (d, J=8.8 Hz, 2H), 7.77 (s, 1H), 7.16 (d, J=8.8 Hz, 2H), 3.66-3.54 (m, 12H), 3.54-3.49 (m, 2H), 3.41 (t, J=6.5 Hz, 2H), 1.75 (m, 2H), 1.57-1.49 (m, 2H), 1.47-1.32 (m, 4H). ¹³C NMR (101 MHz, Acetone) 6143.74, 132.45, 129.94, 119.66, 71.56, 71.10, 71.09, 70.92, 70.76, 70.54, 45.75, 40.50, 38.72, 33.36, 30.28, 27.36, 26.15, HR-ESI-MS: m/z calcd. for 412.1877, measured for 412.1877

UL3 Synthesis

In order to synthesize N-(2-(2-(2-((6-chlorohexyl)oxy)ethoxy)ethoxy)ethyl)-3-(3-methyl-3H-diazirin-3-yl)propenamide (UL3), 3-(3-methyl-3H-diazirin-3-yl)propanoic acid (compound 9, 13.4 mg, 0.05 mmol), EDC-HCl (10.5 mg, 0.055 mmol), TEA (7 μL, 0.05 mmol) and DMAP (6.1 mg, 0.05 mmol) in dried THF (0.3 mL) were mixed in a solution of 2-(2-(2-((6-chlorohexyl)oxy)ethoxy)ethoxy)ethan-1-aminium chloride (1c, 15.2 mg, 0.05 mmol) in THF (0.2 mL). The mixture was stirred for 2 hours and concentrated in vacuum. The crude material was purified by flash chromatography to obtain UL3 (7 mg, yield: 37%).

¹H NMR (600 MHz, Chloroform-d) δ(s, 1H), 3.64-3.51 (m, 12H), 3.48-3.42 (m, 4H), 2.02-1.98 (m, 2H), 1.79-1.72 (m, 4H), 1.60 (p, J=6.9 Hz, 2H), 1.45 (dt, J=14.8, 6.8 Hz, 2H), 1.37 (q, J=8.0 Hz, 2H), 1.02 (s, 3H). ¹³C NMR (150 MHz, Chloroform-d) 671.26, 70.50, 70.47, 70.20, 70.05, 69.82, 45.01, 39.27, 32.50, 30.57, 30.06, 29.38, 26.65, 25.46, 25.37, 19.86. HRMS-DART: m/z calcd for 377.2081, measured for 378.2154 [M+H]⁺.

UL4 Synthesis

In order to synthesize N-(2-(2-(2-((6-chlorohexyl)oxy)ethoxy)ethoxy)ethyl)-4-(3-(trifluoromethyl)-3H-diazirin-3-yl)benzamide (UL4), 4-(3-(trifluoromethyl)-3H-diazirin-3-yl)benzoic acid (compound 10, 11.5 mg, 0.05 mmol), EDC-HCl (10.5 mg, 0.055 mmol), TEA (7 μL, 0.05 mmol) and a solution of DMAP (6.1 mg, 0.05 mmol) in crude THF (0.3 mL) were added to a solution of 2-(2-(2-((6-chlorohexyl)oxy)ethoxy)ethoxy)ethan-1-aminium chloride (15.2 mg, 0.05 mmol) in THF (0.2 mL). The mixture was stirred for 2 hours and concentrated in vacuum. The crude material was purified by flash chromatography to obtain UL4 (14 mg, yield: 60%).

¹H NMR (600 MHz, Chloroform-d) 6-7.82 (m, 2H), 7.23 (d, J=8.2 Hz, 2H), 7.09 (d, J=5.4 Hz, 1H), 3.68-3.61 (m, 10H), 3.54 (dd, J=5.8, 3.7 Hz, 2H), 3.51 (t, J=6.7 Hz, 2H), 3.40 (t, J=6.7 Hz, 2H), 1.78-1.71 (m, 2H), 1.54 (dt, J=14.7, 6.9 Hz, 2H), 1.45-1.38 (m, 2H), 1.32 (p, J=7.6, 7.1 Hz, 2H). ¹³C NMR (100 MHz, Chloroform-d) 6135.70, 132.10, 127.62, 126.46, 126.45, 121.93 (d, J=274.8 Hz), 71.27, 70.54, 70.52, 70.25, 70.05, 69.76, 45.00, 39.91, 32.50, 29.35, 28.36 (d, J=40.5 Hz), 26.64, 25.36. HRMS-DART: m/z calcd for 479.1799, measured for 480.1871 [M+H]⁺.

VL1 Synthesis

3-(6-azido-1,3-dioxo-1H-benzo[de]isoquinolin-2(3H)-yl)propanoic acid (compound 11, 180 mg, 0.582 mmol) was placed in 1 mL of DMF in a 10 mL round bottom flask with HATU (254 mg, 0.67 mmol) and DIPEA (240 μL, 1.3 mmol), and the reaction mixture was stirred at room temperature for 30 minutes until it became a yellow precipitate. Next, NH₂-PEG2-HTL (compound 7) was added with 1 mL of DMF, and the reaction mixture briefly turned to a homogeneous liquid and then precipitated again, and after maintaining stirring for an additional 1 hour, a workup was performed using EtOAc and water. and the organic layer was dried over Na₂SO₄ and purified using a silica gel column (1% MeOH in CHCl₃) to obtain 180 mg of a yellow powder (60%).

¹H NMR (400 MHz, CDCl₃) δ (dd, J=7.3, 1.1 Hz, 1H), 8.59 (d, J=8.0 Hz, 1H), 8.45 (dd, J=8.4, 1.0 Hz, 1H), 7.75 (dd, J=8.4, 7.4 Hz, 1H), 7.48 (d, J=8.0 Hz, 1H), 6.28 (s, 1H), 4.54-4.44 (m, 2H), 3.62-3.38 (m, 11H), 2.71-2.61 (m, 2H), 1.81-1.69 (m, 2H), 1.58 (d, J=7.4 Hz, 2H), 1.48-1.31 (m, 4H). ¹³C NMR (100 MHz, CDCl3) δ163.97, 163.53, 143.73, 132.44, 131.95, 129.28, 129.04, 126.99, 124.46, 122.55, 118.81, 114.80, 77.16, 71.37, 70.35, 70.10, 69.90, 45.15, 39.32, 37.02, 34.86, 32.61, 29.55, 26.79, 25.52.

VL2 Synthesis

3-(6-azido-1,3-dioxo-1H-benzo[de]isoquinolin-2(3H)-yl)propanoic acid (compound 11, 116 mg, 0.37 mmol) was placed in 1 mL of DMF in a 10 mL round bottom flask with HATU (160 mg, 0.42 mmol) and DIPEA (255 μL), and the reaction mixture was stirred at room temperature for 30 minutes until it became a yellow precipitate. Next, NH₂-PEG3-HTL (compound 4) was added with 1 mL of DMF, and the reaction mixture briefly turned to a homogeneous liquid and then precipitated again, and after maintaining stirring for an additional 1 hour, a workup was performed with EtOAc and water. and the organic layer was dried over Na₂SO₄ and purified using a silica gel column (1% MeOH in CHCl₃) to obtain a yellow powder (55%).

¹H NMR (400 MHz, CDCl₃) δ(dd, J=7.3, 1.0 Hz, 1H), 8.59 (d, J=8.0 Hz, 1H), 8.45 (dd, J=8.4, 1.0 Hz, 1H), 7.75 (dd, J=8.3, 7.4 Hz, 1H), 7.48 (d, J=8.0 Hz, 1H), 6.40 (s, 1H), 4.53-4.46 (m, 2H), 3.65-3.53 (m, 9H), 3.50 (t, J=6.7 Hz, 2H), 3.45 (dt, J=11.0, 6.0 Hz, 4H), 2.70-2.61 (m, 2H), 1.79-1.69 (m, 2H), 1.56 (dd, J=14.5, 7.0 Hz, 2H), 1.37 (ddd, J=23.3, 15.9, 8.7 Hz, 4H). ¹³C NMR (101 MHz, CDCl₃) δ170.42, 163.96, 163.51, 143.70, 132.42, 131.93, 129.28, 129.01, 126.98, 124.46, 122.58, 118.85, 114.79, 71.36, 70.63 (d, J=4.7 Hz), 70.26 (d, J=15.8 Hz), 69.95, 45.14, 39.38, 37.04, 34.81, 32.63, 29.52, 26.78, 25.51.

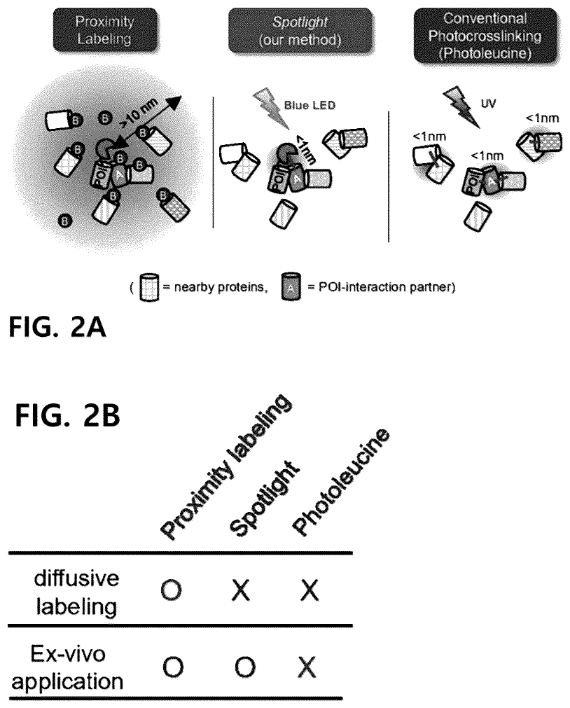

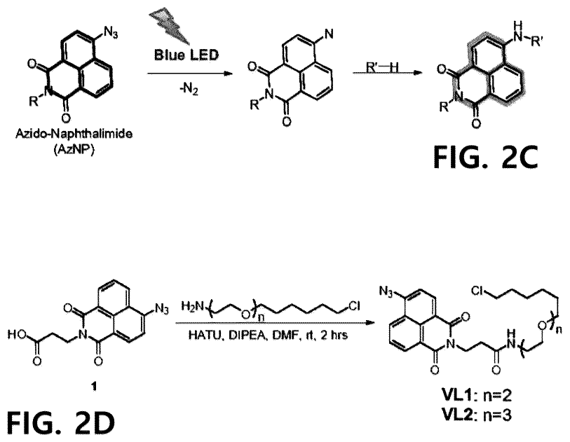

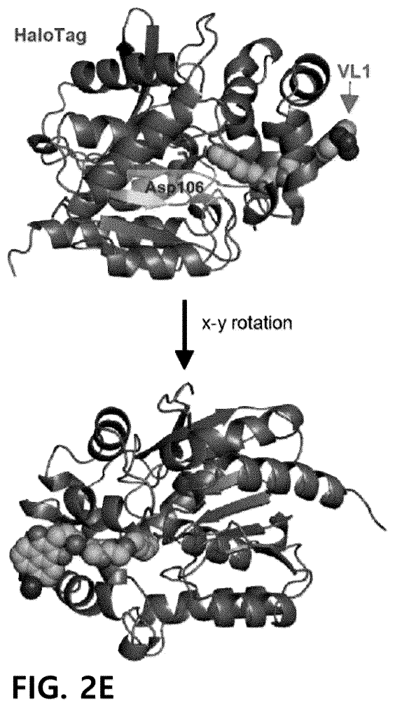

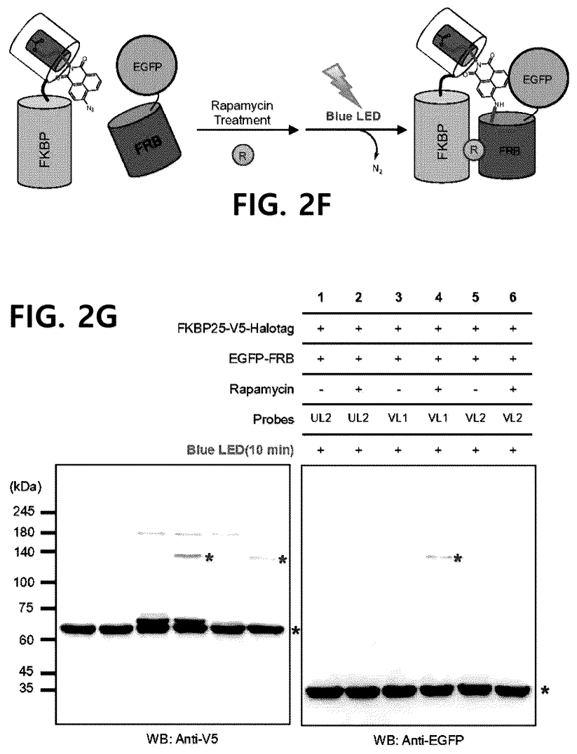

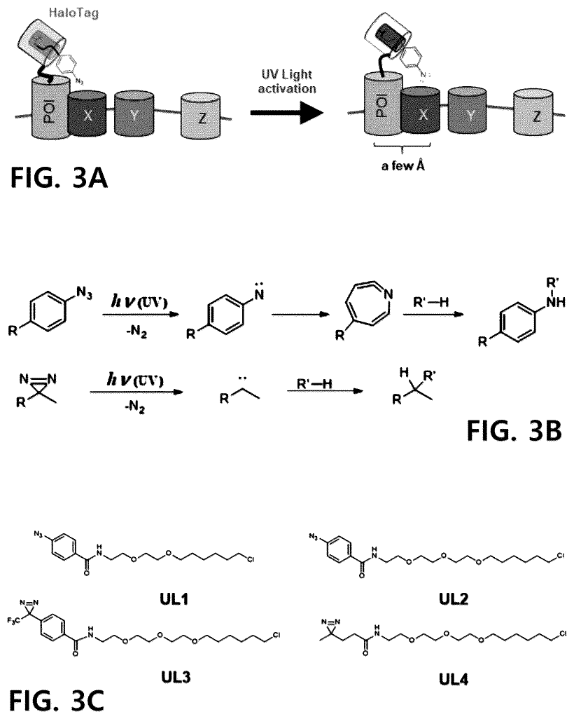

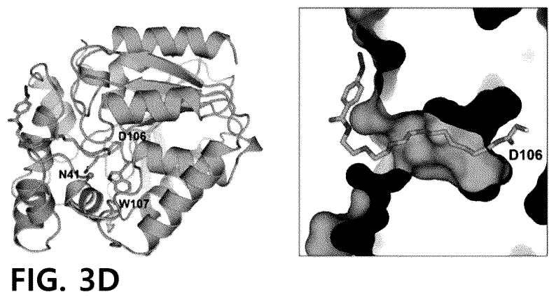

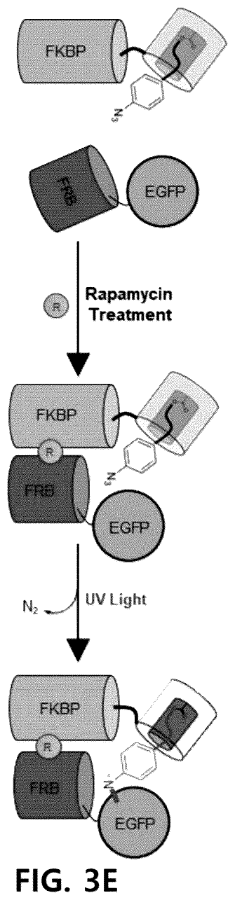

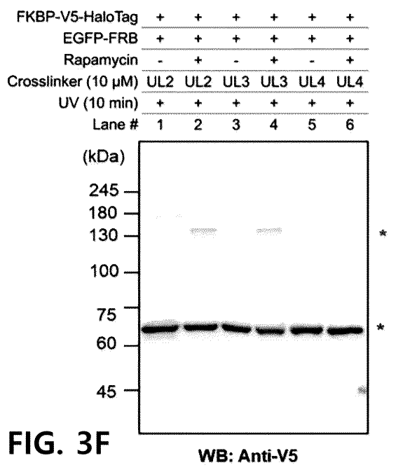

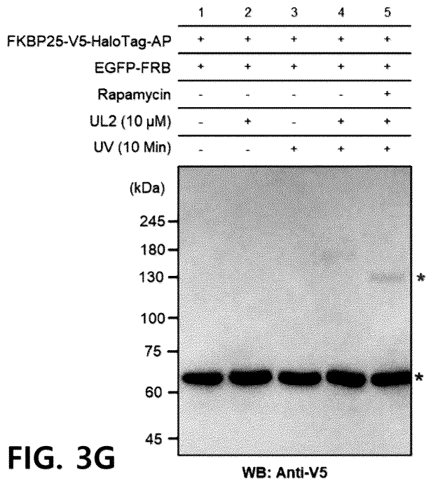

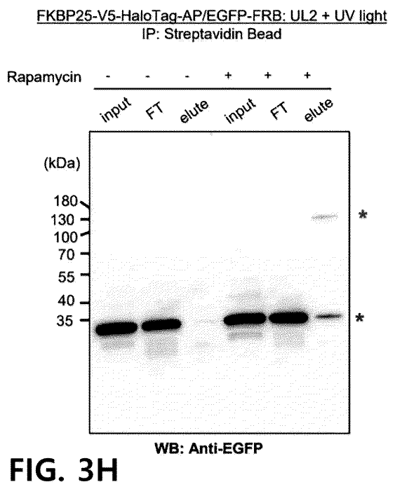

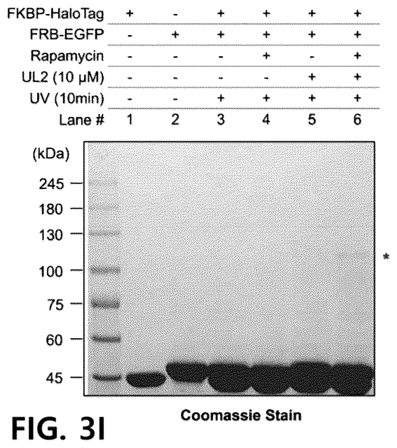

By using X-ray protein structural analysis of the co-crystalized holo-protein complex of HaloTag-UL2 and Halotag-VL1, it was confirmed that the para-azidophenyl moiety of UL2 and the AzNP moiety of VL1 were well exposed on the surface of HaloTag (FIG. 2).

### Example 2. Plasmid Construction and Cloning

Genes were cloned into designated vectors using restriction enzymes and T4 DNA ligase. In order to add a tag (e.g., V5 epitope or AviTag) or signal sequence to the protein, the tag sequence was included in the primers used to PCR amplify the genes. The PCR product was digested with a restriction enzyme and ligated to a vector digested with a restriction enzyme (e.g., pcDNA3, pCDNA5, pDisplay, pET21a and pH6HTN). For expression in mammalian cells, the CMV promoter was used. [Table 1] below shows the plasmids used in the present invention

TABLE 1

| | | Promotor/ | |
|---|---|---|---|
| Name | Features | Vector | Details |
| Nucleocapsid- | KpnI-Nucleocapsid-TEV- | CMV/pCDNA5 | AviTag: GLNDIFEAQKIEWHE |
| Halotag | NheI-V5-Halotag-TEV- | | V5: GKPIPNPLLGLDST |
| | AviTag-Stop-NotI | | TEV: ENLYFQSENLYFQS |
| N-Halo Tag-TurboID | KpnI-Nucleocapsid-TEV- | CMV/pCDNA5 | Flag: DYKDDDDK |
| | NheI-HaloTag-BamHI-V5- | | |
| | TurboID-Flag-NotI | | |
| Halotag-(33aa)- | KpnI-AviTag-NheI-V5- | CMV/ | AviTag: GLNDIFEAQKIEWHE |
| G3BP1 | Halo Tag-BamHI-TEV- | pCDNA5 | V5: GKPIPNPLLGLDST |
| | Flag-G3BP1-Stop-NotI | | TEV: ENLYFQSENLYFQS |
| | | | HA: YPYDVPDYA |
| Halotag-(11 aa)- | HindIII-V5-HaloTag-Flag- | CMV/pCDNA5 | Flag: DYKDDDDK |
| G3BP1 | ClaI-G3BP1-GSG-His6- | | V5: GKPIPNPLLGLDST |
| | Stop | | |
| G3BP1-Halotag | KpnI-G3BP1-BamHI-V5- | CMV/pCDNA5 | V5: GKPIPNPLLGLDST |
| | Halotag-TEV-GSG-His6- | | TEV: ENLYFQSENLYFQS |
| | Stop | | |
| G3BP1-BFP-Halotag | KpnI-G3BP1-BamHI- | CMV/pCDNA5 | Linker: GAPGSAGSAAGSG |
| | Linker-EBFP-AgeI-V5- | | V5: GKPIPNPLLGLDST |
| | Halotag-TEV-His6-Stop- | | TEV: ENLYFQSENLYFQS |
| | XhoI | | |
| FKBP25-V5- | KpnI-FKBP25-BamHI- | CMV/ | V5: GKPIPNPLLGLDST |
| Halo Tag-AviTag | NheI-V5-Halo Tag-Avitag- | pCDNA5 | AviTag: GLNDIFEAQKIEWHE |
| | Stop-NotI | | |
| FKBP12-Halotag- | SpnI-FKBP12-NheI- | CMV/ | V5: GKPIPNPLLGLDST |
| TurboID-Flag | HaloTag-BamHI-V5- | pCDNA5 | Flag:DYKDDDDKDYKDDDDK |
| | TurboID-Flag-NotI | | |
| EGFP-FRB | AgeI-EGFP-HindIII- | CMV/pEGFP | EGFP: Enhanced green fluorescent |
| | EcoRI-FRB-Stop-BamHI | | protein |
| | | | FRB: FK506 rapamycin binding |
| | | | domain of mTOR |
| V5-LaminA/C- | KpnI-AviTag-NheI-V5- | CMV/pCDNA5 | AviTag: GLNDIFEAQKIEWHE |
| Halotag | Halotag--EcoRI- | | V5: GKPIPNPLLGLDST |
| | LaminA/C-Stop-NotI | | |
| Tom20-V5-Halotag | KpnI-Tom20-Linker-NheI- | CMV/pCDNA5 | Linker: GGSGDPPVAT |
| | V5-Halotag-TEV-AviTag- | | AviTag: GLNDIFEAQKIEWHE |
| | Stop-NotI | | V5: GKPIPNPLLGLDST |
| P80Coilin-V5- | KpnI-p80Coilin-BamHI- | CMV/pCDNA5 | AviTag: GLNDIFEAQKIEWHE |
| Halotag | V5-Halotag-TEV-AviTag- | | V5: GKPIPNPLLGLDST |
| | Stop-NotI | | |
| MRPL12-V5-Halotag | KpnI-MRPL12-NheI-V5- | CMV/pCDNA5 | V5: GKPIPNPLLGLDST |
| | Halotag-Strep2-STOP- | | Strep2: WSHPQFEK |
| | NotI | | |
| HNRNPD-V5- | KpnI-HRNPD-NheI- | CMV/pCDNA5 | Linker: GGSG |
| Halotag | Linker-TEV-V5-AViTag- | | |
| | Halotag-His6-Stop-NotI | | |
| V5-Halotag-Sec61b | AflII-V5-Halotag-TEV- | CMV/pCDNA5 | AviTag: GLNDIFEAQKIEWHE |
| | AviTag-NheI-Sec61Beta- | | V5: GKPIPNPLLGLDST |
| | Stop-XhoI | | |
| FKBP12-EGFP | NdeI-FKBP12-HindIII- | T7/pET21a | His6: Six histidine for Ni-NTA |
| | NotI-EGFP-XhoI-His6- | | affinity purification |
| | Stop | | EGFP: Enhanced green fluorescent |
| | | | protein |
| FRB-V5-HaloTag | NotI-His6-FRB-V5-SacII- | T7/pH6HTN | His6: Six histidine for Ni-NTA |
| | Halo Tag-Stop-NotI | | affinity purification |
| | | | FRB: FK506 rapamycin binding |
| | | | domain of mTOR |
| | | | V5: GKPIPNPLLGLDST |
| BirA-His6 | XbaI-BirA-HindIII-NotI- | T7/pET28a | His6: Six histidine for Ni-NTA |
| | His6-Stop | | affinity purification |
| Nucleocapsid-GFP | KpnI-Nucleocapsid-BsiWI- | CMV/pCDNA5 | EGFP: Enhanced green fluorescent |
| | Linker-NheI-EGFP-Stop- | | protein |
| | NotI | | Linker: GAPGSAGSAAGSG |
| EGFP | KpnI-EGFP-Stop-NotI | CMV/pCDNA5 | EGFP: Enhanced green fluorescent |
| | | | protein |
| HA-FRB | HindIII-HA-MluI-FRB- | CMV/PRK5 | FRB: FK506 rapamycin binding |
| | Stop | | domain of mTOR |
| | | | HA: YPYDVPDYA |

### Example 3. Confirmation of Photo-Crosslinking Activity of HaloTag-Specific AzNP Ligand

FKBP25-HaloTag (64 kDa) and EGFP-FRB (48 kDa) were co-expressed in HEK293T cells (ATCC), and the photo-crosslinking activity of the ligand was tested using the rapamycin-inducible FKBP25-FRB protein complex.

HEK293T cells were cultured in DMEM (hyclone, SH30243) supplemented with 10% FBS, 2 mM L-glutamine, 50 units/mL penicillin, and 50 μg/mL streptomycin at 37° C. and 5% CO₂ conditions. In a 12-well plate at 60 to 70% confluency, 1,000 ng of plasmid DNA was mixed with 21 μg of polyethyleneimine (PEI, Polysciences, 23966) using 100 μL no-FBS and DMEM and added to each well, and after 2 to 3 hours, the medium was exchanged with a complete medium. After 24 hours following transfection, cells were treated with DMEM supplemented with 10 μM VL1 for 1 hour. Next, the cells were washed 3 times with DPBS and irradiated with blue LED light for 10 minutes. After removing the DPBS, the cells were lysed with RIPA lysis buffer (Elpis biotech, EBA-1149) at 4° C. for 30 minutes according to the manufacturer's instructions. The sample was then placed on a 6% SDS-PAGE gel and run at 150V for 60 minutes. The protein on the gel was transferred to a nitrocellulose membrane at 400 mA for 90 minutes. Protein loading levels were confirmed by Ponce staining. After blocking with TBST containing 2% skim milk for 1 hour, it was exchanged with a primary antibody (mouse anti-V5 antibody, Invitrogen, Cat. No. R960-25, 1:5,000 dilution or mouse anti-GFP antibody, Invitrogen, Cat No. 33-2600, 1:1,000 dilution) diluted in TBST containing 2% skim milk and incubated for 1 hour. After washing 4 times (5 minutes each) with 1×TBST buffer, it was incubated for 30 minutes with a secondary antibody (goat anti-mouse HRP conjugated antibody, BioRad, Cat. No. 1706516, 1:1,000 dilution) diluted in TB ST containing 2% skim milk. After washing 4 times with 1×TBST buffer, it was developed with an ECL kit (Biorad, 1705061), and images were taken with a Gel doc machine (Genesys).

As a result, it was confirmed that VL1 successfully produced the photo-crosslinking products of FKBP25-HaloTag and EGFP-FRB, which appeared at about 110 to 120 kDa only under the blue LED lighting condition in which rapamycin was added. In this response to FKBP25-HaloTag, VL1 including a PEG1 linker showed stronger crosslinking efficiency with EGFP-FRB compared to VL2 including a PEG2 linker (FIG. 2G).

Meanwhile, in order to verify the above results, even in the results of additional experiments using HA-FRB (13 kDa) and FKBP25-V5-HaloTag, VL1-mediated photo-crosslinking products were identified at about 80 kDa (data not illustrated), which was close to the expected molecular weight of the crosslinked product of HA-FRB and FKBP25-V5-HaloTag.

These results indicate that VL1 selectively crosslinks the interactome of HaloTag-conjugated POI (POI-HaloTag) regardless of the tag protein size of the interaction partner. Meanwhile, none of UL1 to UL4, which produced photo-crosslinked products under UV light illumination, generated photo-crosslinked products under the blue LED illumination condition (FIG. 2G).

### Example 4. Reduction of Proximity Labeling Radius According to TurboID Fusion

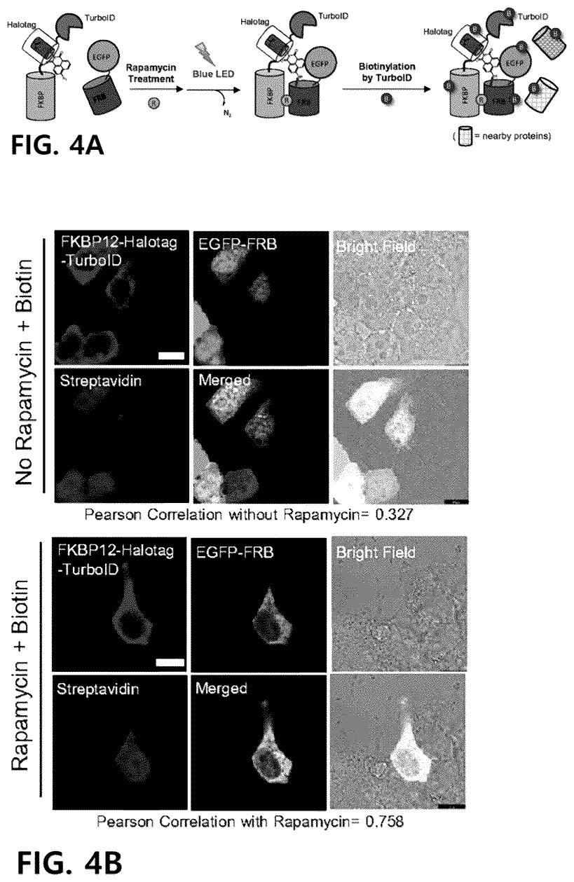

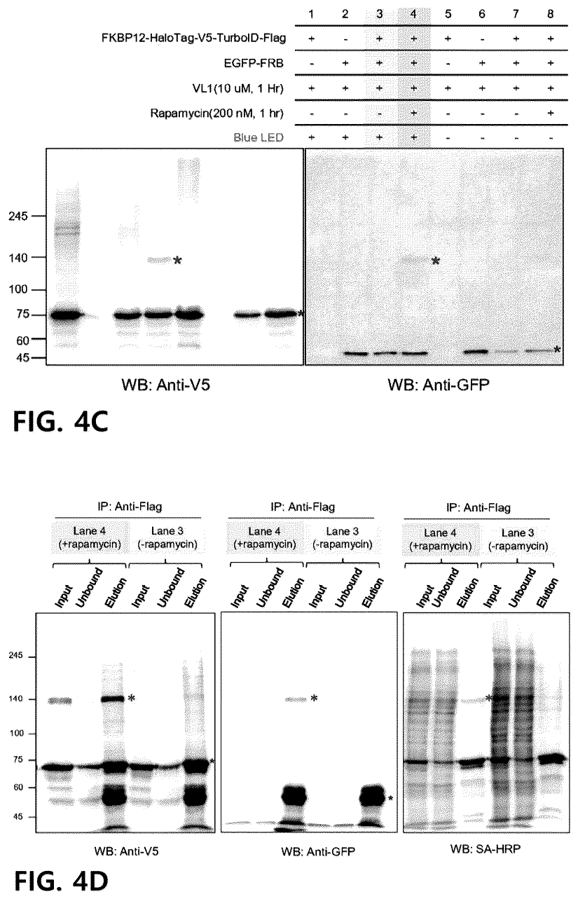

It was attempted to confirm whether FRB could be photo-crosslinked and biotinylated by tagging the FKBP12 protein with HaloTag and TurboID and treating with rapamycin (FIG. 4A).

HEK293T cells were cultured using DMEM supplemented with 10% FBS, 2 mM L-glutamine, 50 unit/mL penicillin and 50 μg/mL streptomycin at 5% CO₂ and 37° C. When cells were 60 to 70% confluent, for 12-well plates, 1,000 ng of plasmid DNA was mixed with 2 μg of polyethyleneimine (PEI, Polysciences, 23966) using 100 μL of no-FBS DMEM and added to a well, and after 2 to 3 hours following the addition, the medium was replaced with a complete medium again. After 24 hours following transfection, 10 μM of VL1 in DMEM was treated intracellularly for 1 hour.

For imaging experiments, cells were washed three times with DPBS (Thermo Fisher, 21300), and the cells were fixed with a 4% paraformaldehyde solution (Chembio, CBPF-9004) in DPBS for 15 minutes at room temperature. The cells were washed twice with DPBS and permeabilized with cold methanol at −20° C. for 5 minutes. The cells were washed twice again with DPBS and blocked with 2% BSA in DPBS (Millipore, 82-100-6) (blocking buffer) at room temperature for 30 minutes. In order to detect the expression of the HaloTag fusion protein, the cells were incubated with mouse anti-V5 antibody (Invitrogen, Cat. No. R960-25, 1:5,000 dilution) for 1 hour at room temperature. After washing 4 times with TBST for 5 minutes each, the cells were incubated concurrently with secondary Alexa Fluor 568-goat anti-mouse IgG (Invitrogen, Cat. No. A-11004, 1:1,000 dilution) for 30 minutes at room temperature.

For the Western blot experiments, cells were washed 3 times with DPBS, irradiated with Blue LED light for 10 minutes, and after DPBS was removed, the RIPA lysis buffer (Elpis Biotech, EBA-1149) was added. The cells were lysed at 4° C. for 30 minutes. Samples were placed on a 6% SDS-PAGE gel and developed at 150V for 60 minutes, and then the proteins separated on the gel were transferred to a nitrocellulose membrane at 400 mA for 90 minutes. The protein loading level was confirmed by Ponce staining, and the Ponce was removed with 1×TBST buffer. It was blocked with 2% skim milk in TBST for 1 hour, and after replacing the blocking solution with a primary antibody (mouse anti-V5 antibody (Invitrogen, Cat. No. R960-25, 1:5,000 dilution)) diluted in 2% skim milk solution, it was incubated for 1 hour. After washing 4 times (5 min each) with 1×TBST buffer, the membranes were incubated with a secondary antibody (goat anti-mouse HRP conjugated antibody, BioRad, Cat. No. 1706516, 1:1,000 dilution) diluted in 2% skim milk solution in TB ST for 30 minutes. After washing 4 times with 1×TBST buffer, it was developed with an ECL kit (Biorad, 1705061), and images were taken with a Gel doc machine (Genesys).

As a result of the imaging experiments, under rapamycin treatment, FKBP12-HaloTag-TurboID was co-localized with EGFP-FRB, which indicates that the conjugated FKBP12 could functionally form a complex with the FRB domain (FIG. 4B). In the Western blot results, the crosslinked products of EGFP-FRB (48 kDa) and FKBP12-HaloTag-V5-TurboID (84 kDa) were found at about 140 kDa only in the rapamycin-treated sample (FIG. 4C), and in lane 1 and lanes 3 and 4, a sharp change in the crosslinking pattern indicates a dramatic change in the protein interactome of FKBP12. After anti-Flag (mouse anti-Flag, Sigma Aldrich Cat. No. F1804) immunoprecipitation, the SA-HRP (Pierce Cat. No, 21130) Western blot results showed that FKBP12 and FRB crosslinked products were biotinylated (FIG. 4D). In addition to these crosslinked FKBP12 and FRB products, numerous proteins biotinylated by TurboID that were not crosslinked with VL1 were detected in the flow-through fraction. This is because the conventional proximity labeling method (i.e., TurboID method) falsely labels not only the interactome but also all nearby proteins present within the labeling radius, whereas the present invention (Spotlight) is a method that more strictly identifies physically interacting proteins (FIG. 4A).

### Example 5. Identification of Protein-Protein Interactions in Intracellular Compartments by Photo-Crosslinked Compounds

The purpose of this study was to test the specificity between the HaloTag protein and the AzNP-conjugated HaloTag ligand (VL1) and whether they can capture a narrow protein-protein interaction (PPI) network in various subcellular compartments of living cells.

Because VL1 emits green fluorescence (λ, =540 nm) and its fluorescence increases after photo-crosslinking reaction, VL1 targeting and formation of POI-HaloTag in living cells can be easily tracked in real time, whereas it was expected that other UV crosslinking ligands (e.g., phenyl azide, diazirine) would not be visualized by microscopic imaging due to negligible fluorescence emission.

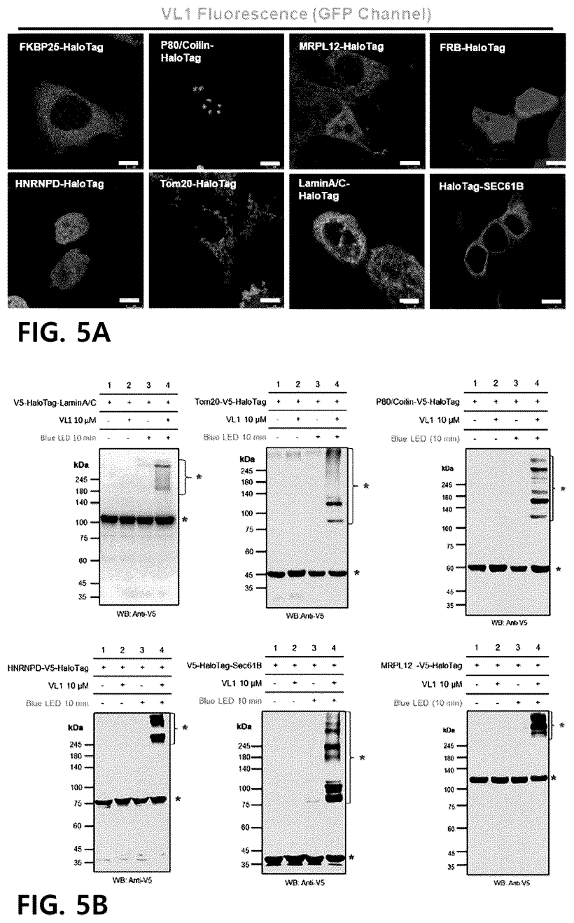

In order to confirm this, the fluorescence image was observed in a manner similar to that described in Example 4, and Western blot experiments were performed using a mouse anti-V5 antibody (Invitrogen, Cat. No. R960-25, 1:5000 dilution). As expected, fluorescence imaging experiments showed that green fluorescent VL1 targeted a variety of HaloTag proteins in various intracellular compartments, including the inner nuclear membrane (Lamin-AC), Cajal body (p80-coilin), SAM68 body (HNRNPD), mitochondrial matrix (MRPL12), mitochondrial outer membrane (Tom20), endoplasmic reticulum membrane (SEC61B) and stress granules (G3BP1) (FIG. 5A).

The Western blot results showed that all of POI-HaloTag structures of various intracellular organelles produced crosslinked products with VL1 under blue LED illumination (FIG. 5B). Interestingly, each POI-HaloTag showed a unique VL1 crosslinking pattern that could reflect the local microenvironment. These results support that VL1 has good membrane permeability and can reach internal parts of organelles, such as mitochondrial matrix protein (i.e., MRPL12), and capture interacting proteins in each compartment with blue LED light activation.

### Example 6. Identification of Spatiotemporal Changes of Interactomes of Photo-Crosslinked Compounds Under Stress Conditions

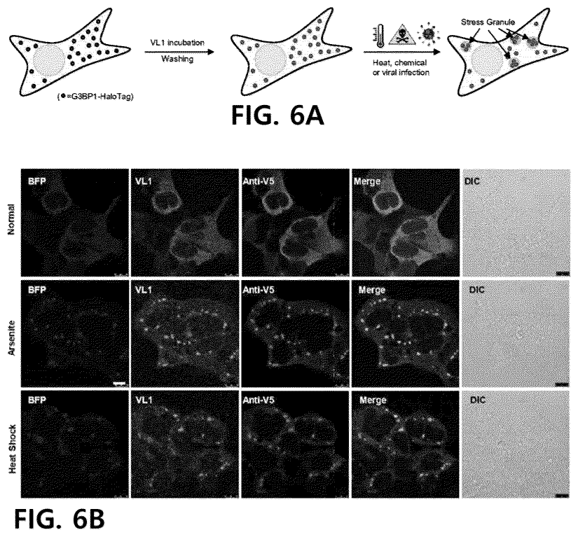

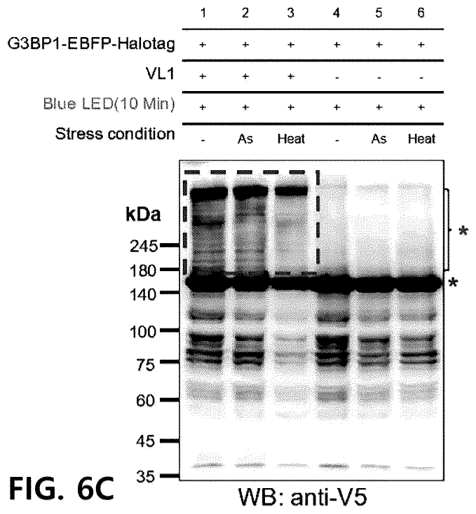

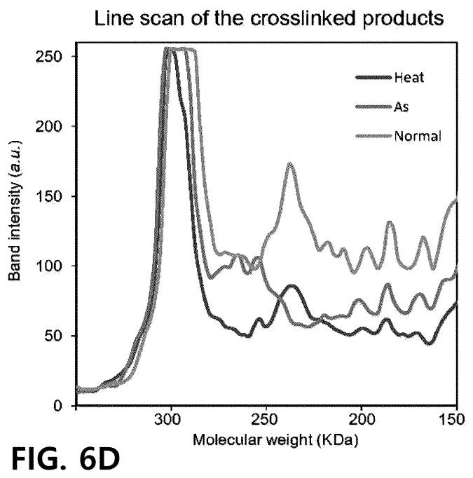

HEK293 T cells expressing HaloTag-EBFP-V5-G3BP1 (hereinafter, HaloTag-G3BP1) were prepared to confirm that VL1 has stability to capture the spatiotemporal changes of the interactome under stress conditions (FIG. 6A).

The cells were incubated with VL1 for 1 hour, washed and stimulated for 1 hour under oxidative stress or thermal stress by treatment with sodium arsenite (As₂O₃, 500 μM) at 43° C. As known in previous studies, HaloTag-G3BP1 formed stress granules (e.g., BFP and anti-V5 antibodies) by G3BP1, and HaloTag-targeted VL1 fluorescence also overlapped well with BFP and anti-V5 signals (FIG. 6B).

These results indicate that VL1 can be used for real-time visualization of a protein of interest (POI) under various stimuli. In addition, by confirming that the VL1-mediated photo-crosslinking product was changed by stress conditions (e.g., sodium arsenite, heat shock), it was confirmed that the present invention can capture the change of the G3BP1 interactome according to the environmental change (FIGS. 6c and 6d).

Meanwhile, changes in the G3BP1 interactome were reproducible in the G3BP1-GFP and GFP-binding protein (GBP)-HaloTag systems (data not illustrated), and therefore, it was found that the GFP tag could be useful for rapid interactome mapping of existing clone libraries of designated target proteins (POIs). The present invention using VL1 for activation in blue LED light showed more crosslinked proteins under UV light compared to using UL2, which is interpreted to be mainly attributed to the superior cell penetration of visible light than UV light.

### Example 7. Identification of Interactomes Using SARS-CoV-2-Nucleocapsid (N) Protein-HaloTag in Living Cells

It was attempted to identify the interactome in the host of ^{SARS-CoV-2}N using the system of the present invention. ^{SARS CoV-2}N is one of the key protein components of viral particles with RNA-binding domains that play an important role in replication and repackaging of the viral genome in host cells, and the study of host proteins that interact with viruses is essential to understanding the mechanisms of viral replication in host cells. Previous research methods for the host interactome of ^{SARS-CoV-2}N we re mainly by affinity purification mass spectrometry (AP-MS) and conventional proximity labeling methods. However, AP-MS data may indicate artificial interaction partners due to dissolution conditions, and the existing proximity labeling method identifies non-physiological interaction partners due to the aforementioned diffusion labeling properties.

Therefore, in the present invention, it is predicted that another important information on the physical interaction of ^{SARS-CoV-2}N can be provided, and in order to identify the host interaction network of ^{SARS-CoV-2}N in human cells, a ^{SARS-CoV-2}N-V5-HaloTag-AP construct (hereinafter, SARS-CoV-2N-HaloTag) was cloned, and a cell line stably expressing the same in HEK293T-rex cells was prepared.

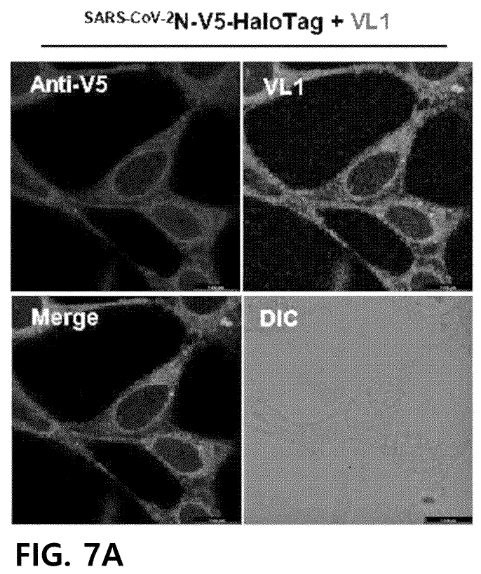

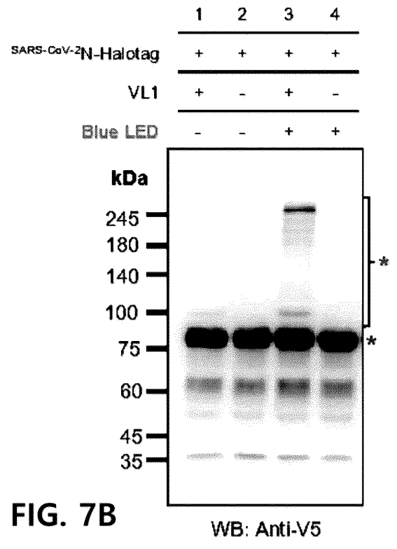

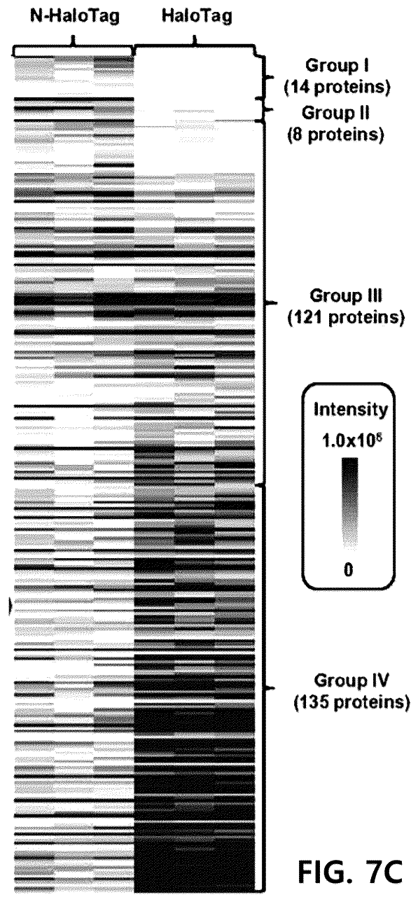

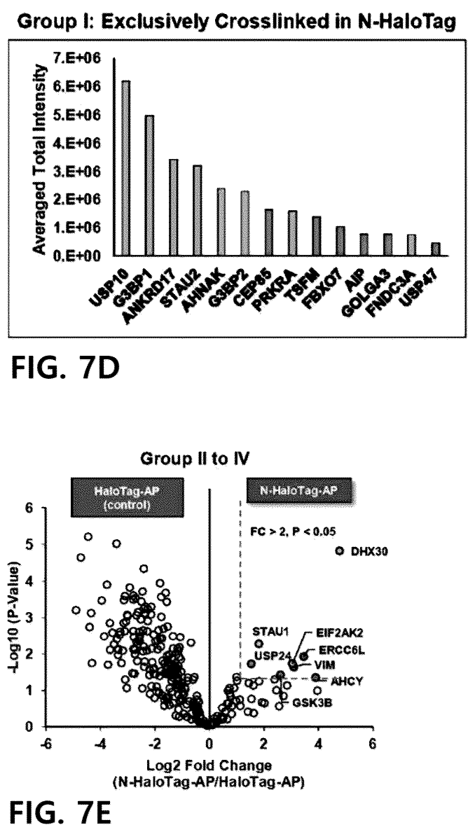

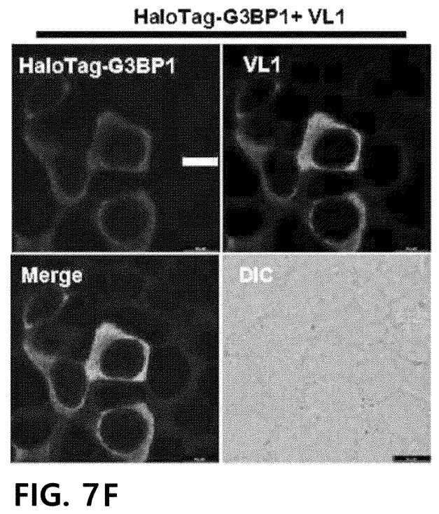

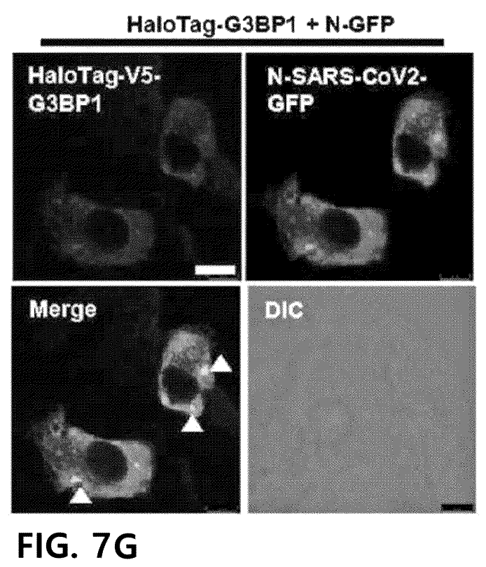

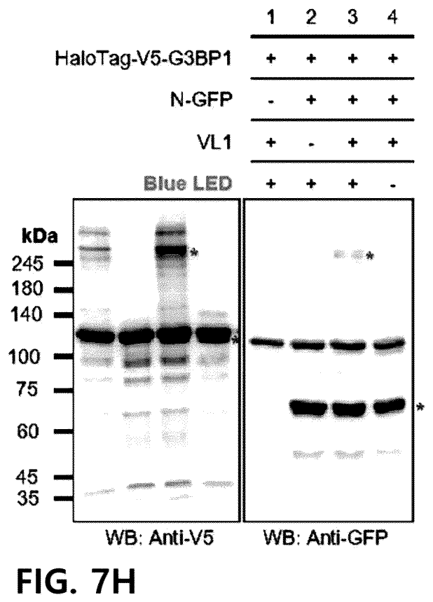

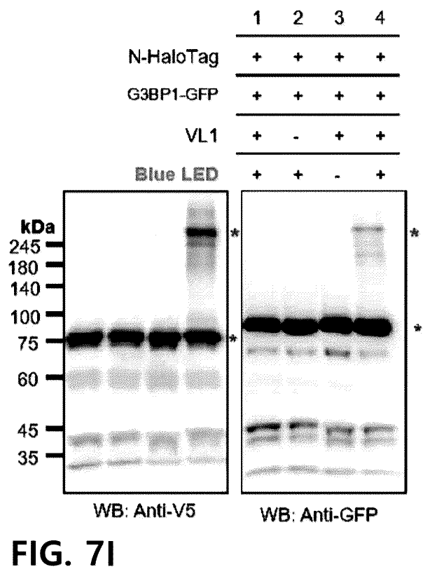

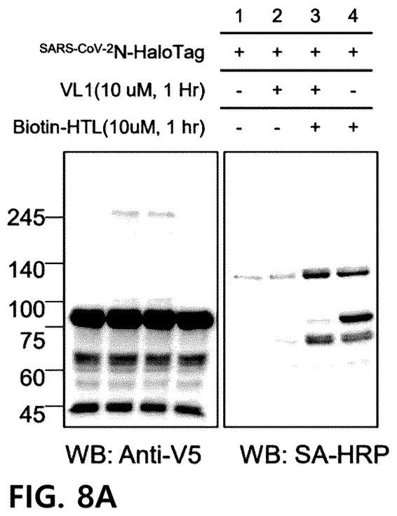

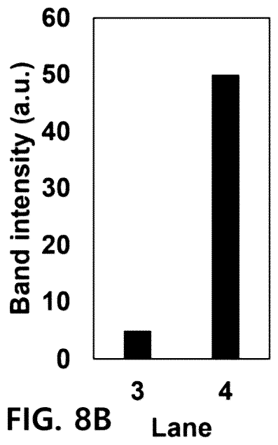

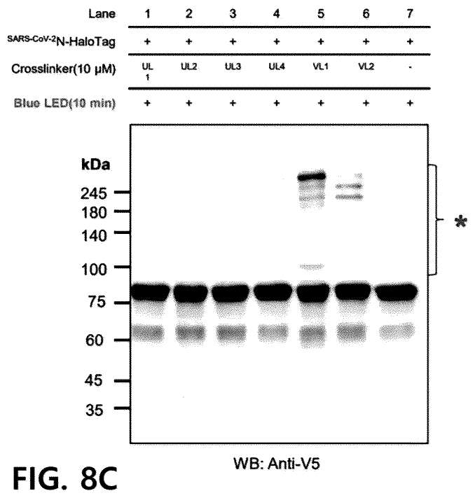

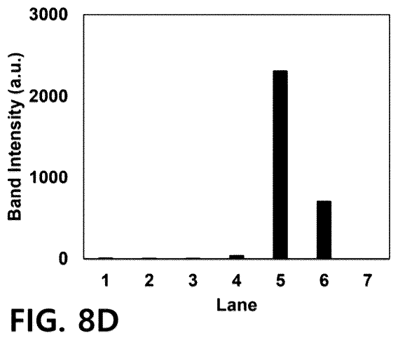

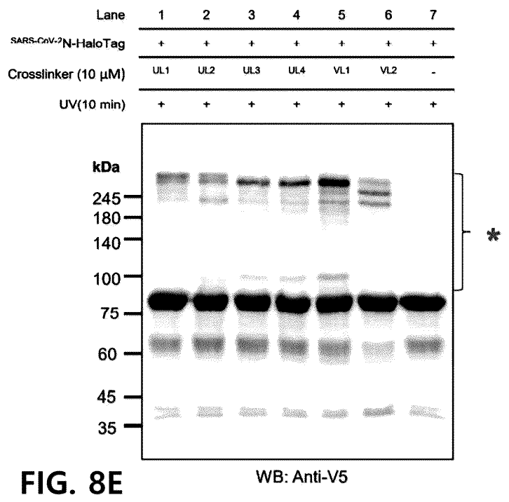

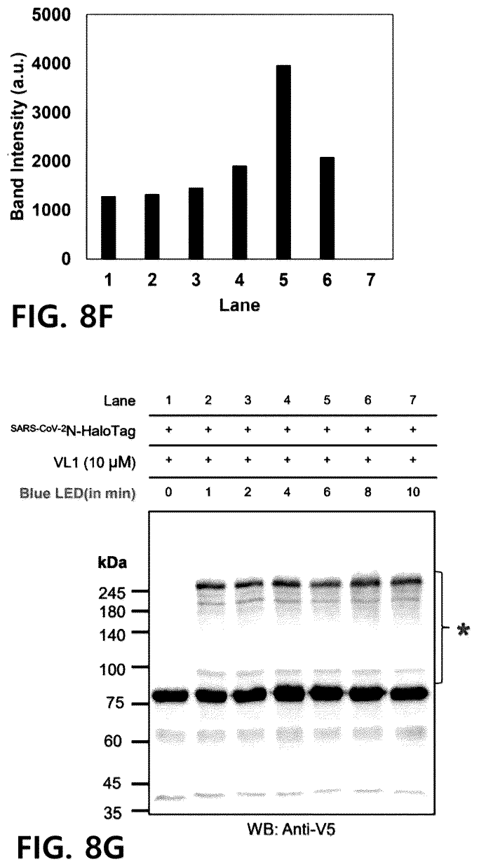

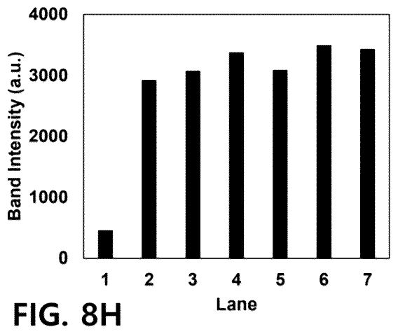

In sequential imaging and Western blot experiments, VL1 successfully targeted the ^{SARS-CoV-2}N-HaloTag protein and crosslinked adjacent proteins (FIGS. 7a and 7b). Moreover, almost all cells expressing ^{SARS-CoV-2}N-HaloTag were labeled with VL1, and therefore, post-treated biotin-HTL was negligibly conjugated with ^{SARS-CoV-2}N-HaloTag after VL1 treatment (FIGS. 8a and 8b). In addition, VL1 showed particularly strong crosslinking efficiency for ^{SARS-CoV-2}N-HaloTag compared to other photo-crosslinking ligands (VL2 and UL probe) under blue LED illumination (FIGS. 8c to 8f), and the photo-crosslinking reaction for ^{SARS-CoV-2}N-HaloTag was efficiently completed within 1 minute after treatment with blue LED illumination. (FIGS. 8g and 8h).

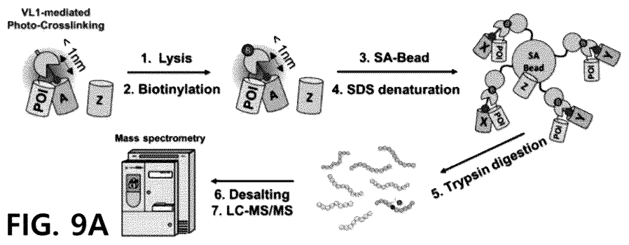

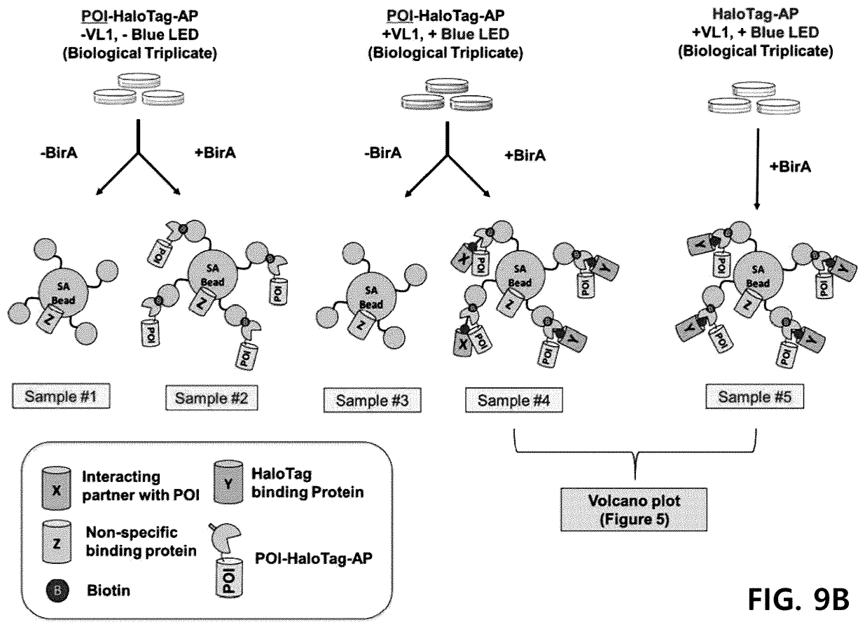

In order to identify the physical interactome of ^{SARS-CoV-2}N in the host cell, triplicate biological samples for LC-MS/MS analysis were prepared for the above composition or control group (FIG. 9).

Specifically, for mass spectrometry of crosslinked products, HEK293T cells were grown to 60 to 70% in 100 Φ plates. 8,000 ng of plasmid DNA was transfected using the PEI transfection reagent, and the medium was replaced with a complete medium after 3 hours. After 24 hours following transfection, it was incubated with 10 μM VL1 for 1 hour and washed 3 times with cold DPBS before crosslinking in blue LED for 10 minutes. Cells were collected in DPBS, the pellet was lysed with RIPA buffer, and the lysate was in vitro-biotinylated using recombinant wild-type BirA solution (10 μM BirA, 10 μM biotin, 200 μM ATP and 500 μM MgCl₂) at room temperature for 12 hours. In order to remove free biotin, the lysate was loaded onto an amicon filter and centrifuged at 12,000×g for 4×15 minutes. 1×TBS buffer was added to up to 400 μL of the concentrated lysate followed by adding 100 μL of washed streptavidin beads. After 10 minutes of incubation, 2 mL of SDS in TBS was added to a final concentration of 2 to 10% and incubated for 1 hour. Beads were washed 4 times with 2% SDS in TBS buffer and incubated with 10 mM DTT in 50 mM ABC buffer for 1 hour at 37° C. Alkylation was performed with 55 mM IAM in ABC buffer at 37° C. under dark conditions. After incubating for 1 hour, the solution was replaced with 200 μL of a trypsin solution (2 μg trypsin and 1 mM CaCl₂ in 50 mM ABC buffer) and mixed at 37° C. for 12 hours. Next, the supernatant was desalted using a zip-tip (Thermo Fisher, 87784), and the eluted fractions were dried using a speed vac and stored in a freezer until loading into LC-MS/MS.

Peptides were analyzed with a Thermo-Scientific Q Exactive Plus equipped with a nanoelectrospray ion source. The peptide mixture was separated by using a C18 reverse-phase HPLC column (500 mm×75 μm i.d.) using a 2.4 to 17.6% acetonitrile/0.1% formic acid gradient at a flow rate of 300 nL/min for 120 minutes. For MS/MS analysis, precursor ion scan MS spectra (m/z 350 to 2,000) were acquired by using an Orbitrap spectrometer at a resolution of 70K at 200 m/z with an internal lock mass. A resolution of 17,500 at m/z 200 was set for the HCD spectrum, and the 15 most concentrated ions were separated and fragmented by higher energy collision dissociation (HCD). For protein identification, MS/MS-based peptide and protein identification was validated by using Scaffold (version 4.11.0, Proteome Software Inc., Portland, OR).

Peptide identification was allowed if it could be established with a probability of 95.0% or more by the Scaffold Local FDR algorithm. Protein identification was allowed if it could be established with a probability of 99.0% or more and it included at least two identified peptides. Protein probabilities were assigned by the Protein Prophet algorithm. Proteins that contained 5 similar peptides and could not be differentiated by MS/MS analysis alone were classified to satisfy the principles of parsimony.

Tandem mass spectra from database searches were extracted by Proteome Discoverer (version 2.2, Thermo Fisher Scientific, San Jose, CA). All MS/MS samples were analyzed using Sequest (XCorr only). Sequest was set up to search the Homo sapiens protein sequence database (42230 entries, UniProt (http://www.uniprot.org/)), assuming digestion with trypsin. Sequest was searched with a fragment ion mass tolerance of 0.80 Da and a parent ion tolerance of 10.0 PPM. The carbamidomethyl of cysteine was designated as a fixed modification in Sequest. The oxidation of methionine and acetyl at the N-terminus was designated as a variable modification in Sequest. For volcano plot analysis, 5 multiple substitutions of missing values were used via PANDA-View Software 6.

In order to concentrate the VL1-crosslinked product of ^{SARS-CoV-2}N-HaloTag, the C-terminus of the ^{SARS-CoV-2}N-HaloTag structure was genetically conjugated with a receptor peptide (AP, also known as Avitag), and after the in vitro biotinylation reaction using purified biotin ligase (BirA), it was allowed to be concentrated in streptavidin (SA) beads. N-HaloTag was biotinylated by adding BirA and biotin/ATP to the mixture. Depending on the femtomolar level of binding affinity between biotin and streptavidin, the biotinylated N-HaloTag after streptavidin-bead concentration did not remain in the flow-through fraction at all or was negligible. This strong binding affinity made it possible to use 10% sodium dodecyl sulfate (SDS) buffer as a culture and wash buffer in the concentration step, which was useful for efficiently removing non-covalent binders of bait proteins. In fact, only 4 proteins (including bait proteins) were significantly concentrated in the ^{SARS-CoV-2}N-HaloTag-AP sample that was not VL1 crosslinked and biotinylated compared to the non-biotinylated sample. In contrast, since 161 proteins were identified in the VL1-SARS-CoV crosslinked and biotinylated ^{SARS-CoV-2}N-HaloTag-AP sample (i.e., +VL1, +BirA), numerous VL1-crosslinking proteins survived with the ^{SARS-CoV-2}N-HaloTag-AP protein.

Due to the possibility that some unknown endogenous protein with intrinsic affinity for HaloTag may also crosslink with VL1, as a control group, a VL1 crosslinked product was generated under blue LED lighting to confirm the VL1 binding affinity for the free HaloTag-AP structure as well.

The HaloTag-AP result according to the same mass spectrometry method provided detailed background protein information, and based on this, the N-interactome could be obtained by filtering the VL1 crosslinked protein information of ^{SARS-CoV-2}N-HaloTag-AP. 14 proprietary proteins (G3BP1, G3BP2, STAU2, AHNAK, ANKRD17, PRKRA, CEP85, FBXO7, FNDC3A, GOLGA3, TSFM AIP, USP10 and USP47) were confirmed in the VL1-crosslinked N-HaloTag sample by comparing MS1 signal intensities in both samples (Group I, FIG. 7D). In addition, the other 8 proteins (ERCC6L, USP24, GSK3B, STAU1, EIF2AK2, VIM, AHCY and DHX30) of the overlapping proteins between N-HaloTag and HaloTag (Groups II to IV) were significantly more abundant in the VL1-crosslinked N-HaloTag sample compared to the VL1-crosslinked HaloTag-AP samples (FIG. 7E). In this relative quantitative analysis, a total of 22 proteins could be selected as “N-interactomes”. 11 of these proteins (USP10, G3BP1, ANKRD17, STAU2, AHNAK, G3BP2, PRKRA, FNDC3A, STAU1, EIF2AK2 and DHX30) are characterized as RBPs. This proportion of RBP in the N-interactomes (50%, 11/22) was significantly higher compared to the human proteome (about 5%, 1072/20,380). In particular, 7 out of 22 proteins (32%) of the N interactomes were also found in stress granules where viral RNA could reside (e.g., G3BP1, G3BP2, STAU1, STAU240, USP10, PRKRA, EIF2AK2 and CEP85), and this data suggests that SARS-CoV-2N does not promiscuously interact with any RBP or any resident protein in the stress granules. Meanwhile, PABPC1 and THRAP3, which are well-known stress granule resident RBPs, were not crosslinked with N-HaloTag-AP but were abundant in samples crosslinked with free HaloTag-AP.

In order to verify that ^{SARS-CoV-2}N and N-interactome are within the VL1-mediated crosslinking radius, G3BP1, which is well known as RBP and a stress granule marker protein, was selected. A HaloTag-G3BP1 construct was constructed in which the entire HaloTag was spliced at the N-terminus of G3BP1. The construct was expressed in the cytoplasm in the basal state, and VL1 anchoring did not change this pattern (FIG. 7F), but when ^{SARS-CoV-2}N-GFP was co-expressed, HaloTag-G3BP1 formed granules and co-localized with ^{SARS-CoV-2}N, which means that G3BP1 and ^{SARS-CoV-2}N are in close proximity (FIG. 7G).

The Western blot results also showed that HaloTag-G3BP1 formed a ^{SARS-CoV-2}N-GFP and VL1-crosslinked product under blue LED illumination and ^{SARS-CoV-2}N-HaloTag also formed a G3BP1-GFP and VL1-crosslinked product (FIG. 7I and FIG. 7H).

As a result of performing anti-Flag IP on the N protein using N-Halotag-TurboID-Flag depending on whether G3BP1-GFP was co-expressed, G3BP1 could be observed in the concentrated fractions after western blot, but GFP signal was not observed in the pass-through fractions, and from these results, it was found that G3BP1 physically interacts with ^{SARS-CoV-2}N. This is also consistent with recent publications showing that G3BP1 is a physical interaction partner based on various validation methods of ^{SARS-CoV-2}N (Gordon, D. E. et al., Nature 2020, 583, 459-468; Nabeel-Shah, S. et al., bioRxiv 2020, 2020.2010.2023.342113).

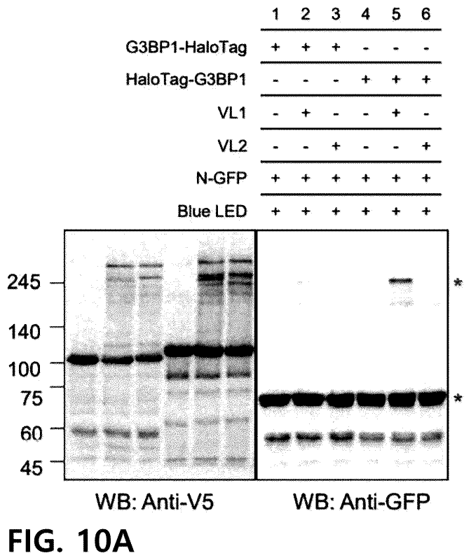

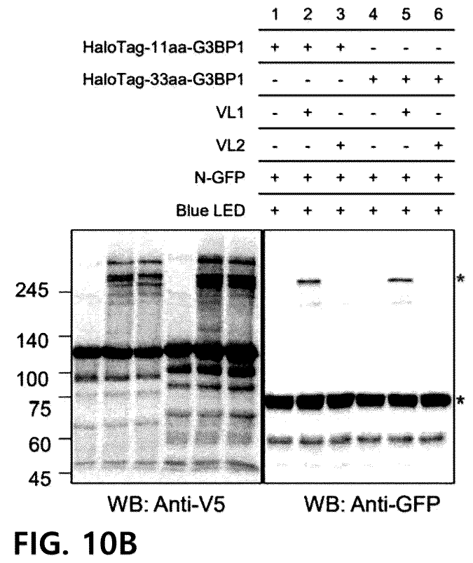

It was further confirmed that the crosslinked product as above was generated specifically for the HaloTag-G3BP1 construct (i.e., not generated in G3BP1-HaloTag) (FIG. 10A), which means that the interaction between G3BP1 and ^{SARS-CoV-2}N takes place in the N-terminal domain of G3BP1. In addition, when the HaloTag-2×TEV-Flag-G3BP1 construct having a longer linker (33 amino acids) than the linker (11 amino acids) used in Halotag-Flag-G3BP1 was used, almost the same crosslinking results were obtained. and it was found that this system was independent of the linker size between HaloTag and POI (FIG. 10B). In contrast, when VL2 (PEG3 linker, 3.43 Å longer than VL1) was used, different crosslinking results were obtained compared to the results obtained using VL1 when it was performed on HaloTag-2×TEV-Flag-G3BP1 or Halotag-Flag-G3BP1 (FIG. 10B).

So far, with respect to the present invention, the preferred exemplary embodiments have been reviewed. Those of ordinary skill in the art to which the present invention pertains will understand that the present invention may be implemented in a modified form without departing from the essential characteristics of the present invention. Therefore, the disclosed exemplary embodiments are to be considered in an illustrative sense rather than a restrictive sense. The scope of the present invention is indicated by the claims rather than the foregoing description, and all differences within the scope equivalent thereto should be construed as being included in the present invention.

### INDUSTRIAL APPLICABILITY

The novel compounds according to the present invention can be photo-crosslinked with a protein or peptide that physically interacts with a target protein or target nucleic acid by the property of being photo-crosslinked to the proximal protein or peptide by irradiation with visible light by binding to the target protein or target nucleic acid, and thus, there are advantages in that it is possible to overcome diffusive labeling, which is a limitation of the conventional proximity labeling technology, by photo-crosslinking by visible light irradiation, which is a safe method for a living body, and to improve the accuracy of protein interactome identification.

1-15. (canceled)

16. A compound represented by [Chemical Formula 1] below: 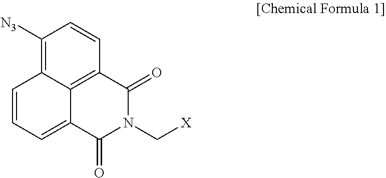
wherein in [Chemical Formula 1] above,
X is C, N, O, S, Si or represented by [Chemical Formula 2] below, 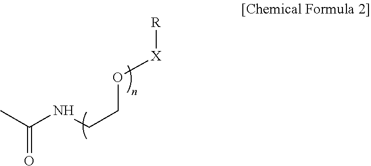
wherein in [Chemical Formula 2] above,
n is an integer of 0 to 10,
X is straight-chain or branched —(CH₂)m-, H or halogen, and m is an integer of 1 to 20, and
R is H, halogen or —CH₃.

17. The compound of claim 16, wherein the compound is a compound represented by [Chemical Formula 3] below: 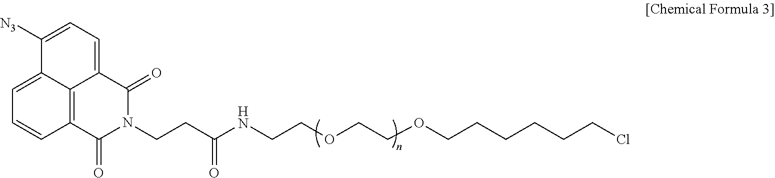
wherein in [Chemical Formula 3] above, n is an integer of 1 to 5.

18. The compound of claim 16, wherein the compound has protein-binding ability.

19. The compound of claim 16, wherein the compound bound to a target protein or target nucleic acid is photo-crosslinked to a protein or peptide that interacts with the target protein or target nucleic acid.

20. The compound of claim 19, wherein the target protein is an antibody or peptide.

21. The compound of claim 19, wherein the target nucleic acid is DNA or RNA.

22. The compound of claim 19, wherein the compound is photo-crosslinked to a protein or peptide that interacts with the target protein or target nucleic acid by irradiation with visible light.

23. The compound of claim 19, wherein the radius of the photo-crosslinking is 0.01 to 1 nm.

24. A method for photo-crosslinking a target protein or target nucleic acid with an interacting protein or peptide in proximity, comprising:
(a) treating a compound, which is bound to a target protein or target nucleic acid, in a sample including a protein or peptide interacting with the target protein or target nucleic acid; and
(b) irradiating visible light,
wherein the compound is represented by [Chemical Formula 1] below: 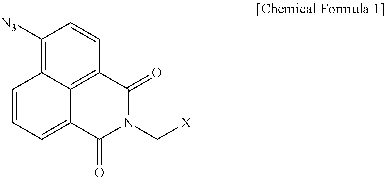
wherein in [Chemical Formula 1] above,
X is C, N, O, S, Si or represented by [Chemical Formula 2] below, 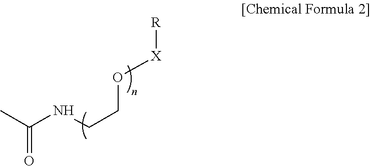
wherein in [Chemical Formula 2] above,
n is an integer of 0 to 10,
X is straight-chain or branched —(CH₂)m-, H or halogen, and m is an integer of 1 to 20, and
R is H, halogen or —CH₃.

25. The method of claim 24, wherein the target protein or target nucleic acid is conjugated to the compound through (i) an internally existing amine group or an externally introduced amine group, or (ii) an internally existing thiol group or an externally introduced thiol group.

26. The method of claim 24, wherein the target protein or target nucleic acid is conjugated with a protein tag.

27. A method for identifying an interacting protein of a target protein, comprising the steps of:
(a) expressing a target protein in a cell;
(b) treating the cell with the compound of claim 16;
(c) irradiating visible light to the cell; and
(d) lysing the cell to identify the protein that is photo-crosslinked with the compound as an interacting protein of the target protein.

28. The method of claim 27, wherein the target protein is conjugated with an epitope peptide tag.

29. The method of claim 28, wherein in step (d), the target protein is precipitated with an epitope peptide-specific antibody to identify the protein that is photo-crosslinked with the compound as an interacting protein of the target protein.

30. The method of claim 27, wherein step (d) is identified by mass spectrometry.


SEQUENCE LISTING (50 sequences)

SEQ ID NO: 1 - protein, 15 aa, Artificial Sequence
  AviTag
GLNDIFEAQKIEWHE

SEQ ID NO: 2 - protein, 14 aa, Artificial Sequence
  V5
GKPIPNPLLGLDST

SEQ ID NO: 3 - protein, 14 aa, Artificial Sequence
  TEV
ENLYFQSENLYFQS

SEQ ID NO: 4 - protein, 8 aa, Artificial Sequence
  Flag
DYKDDDDK

SEQ ID NO: 5 - protein, 9 aa, Artificial Sequence
  HA
YPYDVPDYA

SEQ ID NO: 6 - protein, 13 aa, Artificial Sequence
  Linker
GAPGSAGSAAGSG

SEQ ID NO: 7 - protein, 10 aa, Artificial Sequence
  Linker
GGSGDPPVAT

SEQ ID NO: 8 - protein, 8 aa, Artificial Sequence
  Strep2
WSHPQFEK

SEQ ID NO: 9 - protein, 4 aa, Artificial Sequence
  Linker
GGSG

SEQ ID NO: 10 - protein, 562 aa, Artificial Sequence
  FKBP25-V5-HaloTag (64kDa)
MAAAVPQRAWTVEQLRSEQLPKKDIIKFLQEHGSDSFLAEHKLLGNIKNVAKTANKDHLVTAYNHLFETKRFKGTESISKVSEQVKNVKLNEDKPKETKSEETLDEGPPKYTKSVLKKGDKTNFPKKGDVVHCWYTGTLQDGTVFDTNIQTSAKKKKNAKPLSFKVGVGKVIRGWDEALLTMSKGEKARLEIEPEWAYGKKGQPDAKIPPNAKLTFEVELVDIDKDPLAGKPIPNPLLGLDSTMAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGENLYFQSGLNDIFEAQKIEWHE

SEQ ID NO: 11 - DNA, 31 bp, Artificial Sequence
  Forward Primer for FKBP25-V5-HaloTag (64kDa)
tagtagggtaccatggcggcggccgttccac

SEQ ID NO: 12 - DNA, 52 bp, Artificial Sequence
  Reverse Primer for FKBP25-V5-HaloTag (64kDa)
tagtaggctagcggatccttatcaatatccactaattccacttcaaaagtga

SEQ ID NO: 13 - protein, 352 aa, Artificial Sequence
  EGFP-FRB (48kDa)
MASRILWHEMWHEGLEEASRLYFGERNVKGMFEVLEPLHAMMERGPQTLKETSFNQAYGRDLMEAQEWCRKYMKSGNVKDLLQAWDLYYHVFRRISKTSKLAAAVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIDDGSVQLADHYQQNTPIGDGPVLLPDNHYLSTQSALSKDPNEKRDHMVLLEFVTAAGITLGMDELYKGGSGGSGGSR

SEQ ID NO: 14 - DNA, 48 bp, Artificial Sequence
  Forward primer for EGFP-FRB (48kDa)
tagtaggcggccgccaccatggcttctagaatcctctggcatgagatg

SEQ ID NO: 15 - DNA, 45 bp, Artificial Sequence
  Reverse primer for EGFP-FRB (48kDa)
tagtagctcgagtcattatctagatccaccagatccaccagatcc

SEQ ID NO: 16 - protein, 224 aa, Artificial Sequence
  FKBP25
MAAAVPQRAWTVEQLRSEQLPKKDIIKFLQEHGSDSFLAEHKLLGNIKNVAKTANKDHLVTAYNHLFETKRFKGTESISKVSEQVKNVKLNEDKPKETKSEETLDEGPPKYTKSVLKKGDKTNFPKKGDVVHCWYTGTLQDGTVFDTNIQTSAKKKKNAKPLSFKVGVGKVIRGWDEALLTMSKGEKARLEIEPEWAYGKKGQPDAKIPPNAKLTFEVELVDID

SEQ ID NO: 17 - protein, 99 aa, Artificial Sequence
  FRB
MASRILWHEMWHEGLEEASRLYFGERNVKGMFEVLEPLHAMMERGPQTLKETSFNQAYGRDLMEAQEWCRKYMKSGNVKDLLQAWDLYYHVFRRISKTS

SEQ ID NO: 18 - protein, 297 aa, Artificial Sequence
  HaloTag
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 19 - protein, 757 aa, Artificial Sequence
  FKBP12-HaloTag-TurboID-Flag
MGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEKLAMAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGKDPGKPIPNPLLGLDSTKDNTVPLKLIALLANGEFHSGEQLGETLGMSRAAINKHIQTLRDWGVDVFTVPGKGYSLPEPIPLLNAKQILGQLDGGSVAVLPVVDSTNQYLLDRIGELKSGDACIAEYQQAGRGSRGRKWFSPFGANLYLSMFWRLKRGPAAIGLGPVIGIVMAEALRKLGADKVRVKWPNDLYLQDRKLAGILVELAGITGDAAQIVIGAGINVAMRRVEESVVNQGWITLQEAGINLDRNTLAATLIRELRAALELFEQEGLAPYLPRWEKLDNFINRPVKLIIGDKEIFGISRGIDKQGALLLEQDGVIKPWMGGEISLRSAEKDYKDDDDKAAARV

SEQ ID NO: 20 - DNA, 37 bp, Artificial Sequence
  Forward primer for FKBP12-HaloTag-TurboID-Flag
tagtagggtaccatgggagtgcaggtggaaaccatct

SEQ ID NO: 21 - DNA, 45 bp, Artificial Sequence
  Reverse primer for FKBP12-HaloTag-TurboID-Flag
tagtaggctagcttttccagttttagaagctccacatcgaagacg

SEQ ID NO: 22 - protein, 108 aa, Artificial Sequence
  FKBP12
MGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 23 - protein, 320 aa, Artificial Sequence
  TurboID
MKDNTVPLKLIALLANGEFHSGEQLGETLGMSRAAINKHIQTLRDWGVDVFTVPGKGYSLPEPIPLLNAKQILGQLDGGSVAVLPVVDSTNQYLLDRIGELKSGDACIAEYQQAGRGSRGRKWFSPFGANLYLSMFWRLKRGPAAIGLGPVIGIVMAEALRKLGADKVRVKWPNDLYLQDRKLAGILVELAGITGDAAQIVIGAGINVAMRRVEESVVNQGWITLQEAGINLDRNTLAATLIRELRAALELFEQEGLAPYLPRWEKLDNFINRPVKLIIGDKEIFGISRGIDKQGALLLEQDGVIKPWMGGEISLRSAEK

SEQ ID NO: 24 - protein, 997 aa, Artificial Sequence
  Lamin-AC-V5-HaloTag
MGLNDIFEAQKIEWHEASGKPIPNPLLGLDSTMAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGEFLKMETPSQRRATRSGAQASSTPLSPTRITRLQEKEDLQELNDRLAVYIDRVRSLETENAGLRLRITESEEVVSREVSGIKAAYEAELGDARKTLDSVAKERARLQLELSKVREEFKELKARNTKKEGDLIAAQARLKDLEALLNSKEAALSTALSEKRTLEGELHDLRGQVAKLEAALGEAKKQLQDEMLRRVDAENRLQTMKEELDFQKNIYSEELRETKRRHETRLVEIDNGKQREFESRLADALQELRAQHEDQVEQYKKELEKTYSAKLDNARQSAERNSNLVGAAHEELQQSRIRIDSLSAQLSQLQKQLAAKEAKLRDLEDSLARERDTSRRLLAEKEREMAEMRARMQQQLDEYQELLDIKLALDMEIHAYRKLLEGEEERLRLSPSPTSQRSRGRASSHSSQTQGGGSVTKKRKLESTESRSSFSQHARTSGRVAVEEVDEEGKFVRLRNKSNEDQSMGNWQIKRQNGDDPLLTYRFPPKFTLKAGQVVTIWAAGAGATHSPPTDLVWKAQNTWGCGNSLRTALINSTGEEVAMRKLVRSVTVVEDDEDEDGDDLLHHHHGSHCSSSGDPAEYNLRSRTVLCGTCGQPADKASASGSGAQVGGPISSGSSASSVTVTRSYRSVGGSGGGSFGDNLVTRSYLLGNSSPRTQSPQNCSIM

SEQ ID NO: 25 - DNA, 34 bp, Artificial Sequence
  Forward primer for Lamin-AC-V5-HaloTag
tagtagggtaccatgggcctgaacgacatcttcg

SEQ ID NO: 26 - DNA, 40 bp, Artificial Sequence
  Reverse primer for Lamin-AC-V5-HaloTag
tagtaggcggccgctcattacatgatgctgcagttctggg

SEQ ID NO: 27 - protein, 914 aa, Artificial Sequence
  P80-coilin-V5-HaloTag
MAASETVRLRLQFDYPPPATPHCTAFWLLVDLNRCRVVTDLISLIRQRFGFSSGAFLGLYLEGGLLPPAESARLVRDNDCLRVKLEERGVAENSVVISNGDINLSLRKAKKRAFQLEEGEETEPDCKYSKKHWKSRENNNNNEKVLDLEPKAVTDQTVSKKNKRKNKATCGTVGDDNEEAKRKSPKKKEKCEYKKKAKNPKSPKVQAVKDWANQRCSSPKGSARNSLVKAKRKGSVSVCSKESPSSSSESESCDESISDGPSKVTLEARNSSEKLPTELSKEEPSTKNTTADKLAIKLGFSLTPSKGKTSGTTSSSSDSSAESDDQCLMSSSTPECAAGFLKTVGLFAGRGRPGPGLSSQTAGAAGWRRSGSNGGGQAPGASPSVSLPASLGRGWGREENLFSWKGAKGRGMRGRGRGRGHPVSCVVNRSTDNQRQQQLNDVVKNSSTIIQNPVETPKKDYSLLPLLAAAPQVGEKIAFKLLELTSSYSPDVSDYKEGRILSHNPETQQVDIEILSSLPALREPGKFDLVYHNENGAEVVEYAVTQESKITVFWKELIDPRLIIESPSNTSSTEPAKDPLAGKPIPNPLLGLDSTMAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGENLYFQSGLNDIFEAQKIEWHE

SEQ ID NO: 28 - DNA, 42 bp, Artificial Sequence
  Forward primer for P80-coilin-V5-HaloTag
tagtagaagcttggtaccatggcagcttccgagacggttagg

SEQ ID NO: 29 - DNA, 47 bp, Artificial Sequence
  Reverse primer for P80-coilin-V5-HaloTag
tagtaggctagcggatccttggcaggttctgtacttgatgtgttact

SEQ ID NO: 30 - protein, 701 aa, Artificial Sequence
  HNRNPD-V5-HaloTag
MSEEQFGGDGAAAAATAAVGGSAGEQEGAMVAATQGAAAAAGSGAGTGGGTASGGTEGGSAESEGAKIDASKNEEDEGHSNSSPRHSEAATAQREEWKMFIGGLSWDTTKKDLKDYFSKFGEVVDCTLKLDPITGRSRGFGFVLFKESESVDKVMDQKEHKLNGKVIDPKRAKAMKTKEPVKKIFVGGLSPDTPEEKIREYFGGFGEVESIELPMDNKTNKRRGFCFITFKEEEPVKKIMEKKYHNVGLSKCEIKVAMSKEQYQQQQQWGSRGGFAGRARGRGGGPSQNWNQGYSNYWNQGYGNYGYNSQGYGGYGGYDYTGYNNYYGYGDYSNQQSGYGKVSRRGGHQNSYKPYKLAGGSGENLYFQSGKPIPNPLLGLDSTGLNDIFEAQKIEWHEMAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGHHHHHH

SEQ ID NO: 31 - DNA, 34 bp, Artificial Sequence
  Forward primer for HNRNPD-V5-HaloTag
tagtagggtaccatgtcggaggagcagttcggcg

SEQ ID NO: 32 - DNA, 42 bp, Artificial Sequence
  Reverse primer for HNRNPD-V5-HaloTag
gctagcttgtatggtttgtagctattttgatgaccacctcgc

SEQ ID NO: 33 - protein, 522 aa, Artificial Sequence
  MRPL12-V5-HaloTag
MLPAAARPLWGPCLGLRAAAFRLARRQVPCVCAVRHMRSSGHQRCEALAGAPLDNAPKEYPPKIQQLVQDIASLTLLEISDLNELLKKTLKIQDVGLVPMGGVMSGAVPAAAAQEAVEEDIPIAKERTHFTVRLTEAKPVDKVKLIKEIKNYIQGINLVQAKKLVESLPQEIKANVAKAEAEKIKAALEAVGGTVVLEKDPLAGKPIPNPLLGLDSTMAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGWSHPQFEK

SEQ ID NO: 34 - DNA, 37 bp, Artificial Sequence
  Forward primer for MRPL12-V5-HaloTag
tagtagaagcttggtaccatgctgccggcggccgctc

SEQ ID NO: 35 - DNA, 40 bp, Artificial Sequence
  Reverse primer for MRPL12-V5-HaloTag
tagtaggctagcggatccttctccagaaccacggtgccgc

SEQ ID NO: 36 - protein, 501 aa, Artificial Sequence
  Tom20-V5-HaloTag
MVGRNSAIAAGVCGALFIGYCIYFDRKRRSDPNFKNRLRERRKKQKLAKERAGLSKLPDLKDAEAVQKFFLEEIQLGEELLAQGEYEKGVDHLTNAIAVCGQPQQLLQVLQQTLPPPVFQMLLTKLPTISQRIVSAQSLAEDDVEGGSGDPPVATKLAGGSGGSGGSRGKPIPNPLLGLDSTMAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGENLYFQSGLNDIFEAQKIEWHE

SEQ ID NO: 37 - DNA, 32 bp, Artificial Sequence
  Forward primer for Tom20-V5-HaloTag
tagtaggctagcaggcaagcccatccccaacc

SEQ ID NO: 38 - DNA, 106 bp, Artificial Sequence
  Reverse primer for Tom20-V5-HaloTag
tagtaggcggccgctcattactcgtgccactcgatcttctgggcctcgaagatgtcgttcaggccagactgaaaatacaggttttcgccggaaatctcgagcgtcg

SEQ ID NO: 39 - protein, 433 aa, Artificial Sequence
  SEC61B-V5-HaloTag
MGKPIPNPLLGLDSTMAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGENLYFQSGLNDIFEAQKIEWHEKLAMPGPTPSGTNVGSSGRSPSKAVAARAAGSTVRQRKNASCGTRSAGRTTSAGTGGMWRFYTEDSPGLKVGPVPVLVMSLLFIASVFMLHIWGKYTRS

SEQ ID NO: 40 - DNA, 32 bp, Artificial Sequence
  Forward primer for SEC61B-V5-HaloTag
tagtagcttaagatgggcaagcccatccccaa

SEQ ID NO: 41 - DNA, 36 bp, Artificial Sequence
  Reverse primer for SEC61B-V5-HaloTag
tagtaggctagcttctcgtgccactcgatcttctgg

SEQ ID NO: 42 - protein, 822 aa, Artificial Sequence
  G3BP1-V5-HaloTag
MGLNDIFEAQKIEWHEASGKPIPNPLLGLDSTMAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGSENLYFQSENLYFQSDYKDDDDKELSMVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQ

SEQ ID NO: 43 - DNA, 33 bp, Artificial Sequence
  Forward primer for G3BP1-V5-HaloTag
tagtagaagcttggtaccatgggcctgaacgac

SEQ ID NO: 44 - DNA, 35 bp, Artificial Sequence
  Reverse primer for G3BP1-V5-HaloTag
tagtaggcggccgctcattactgccgtggcgcaag

SEQ ID NO: 45 - protein, 1066 aa, Artificial Sequence
  HaloTag-EBFP-V5-G3BP1
MVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQKDPGAPGSAGSAAGSGVSKGEELFTGVVPILVELDGDVNGHKFSVRGEGEGDATNGKLTLKFICTTGKLPVPWPTLVTTLSHGVQCFARYPDHMKQHDFFKSAMPEGYVQERTIFFKDDGTYKTRAEVKFEGDTLVNRIELKGVDFKEDGNILGHKLEYNFNSHNIYIMAVKQKNGIKVNFKIRHNVEDGSVQLADHYQQNTPIGDGPVLLPDSHYLSTQSVLSKDPNEKRDHMVLLEFRTAAGITLGMDELYKKLAGGSGENLYFQSGKPIPNPLLGLDSTGLNDIFEAQKIEWHEMAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGHHHHHH

SEQ ID NO: 46 - DNA, 34 bp, Artificial Sequence
  Forward primer for HaloTag-EBFP-V5-G3BP1
tagtagaagcttggtaccatggtgatggagaagc

SEQ ID NO: 47 - DNA, 39 bp, Artificial Sequence
  Reverse primer for HaloTag-EBFP-V5-G3BP1
tagtaggctagcttcttgtacagctcgtccatgccgaga

SEQ ID NO: 48 - protein, 769 aa, Artificial Sequence
  SARS-CoV-2N-V5-HaloTag-AP
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAENLYFQSENLYFQSKLAGKPIPNPLLGLDSTMAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGENLYFQSGLNDIFEAQKIEWHE

SEQ ID NO: 49 - DNA, 41 bp, Artificial Sequence
  Forward primer for SARS-CoV-2N-V5-HaloTag-AP
tagtagggtaccatgtctgataatggaccccaaaatcagcg

SEQ ID NO: 50 - DNA, 79 bp, Artificial Sequence
  Reverse primer for SARS-CoV-2N-V5-HaloTag-AP
tagtaggctagcttagactgaaaatacaggttttcagactgaaaatacaggttttcggcctgagttgagtcagcactgc